(19) United States
(12) Patent Application Publication
(10) Pub. No.: US 2015/0366944 A1
(43) Pub. Date: Dec. 24, 2015

(54) INTERFERON TREATMENT TARGETING MUTANT P53 EXPRESSING CELLS

(71) Applicant: YEDA RESEARCH AND DEVELOPMENT CO. LTD., Rehovot (IL)

(72) Inventors: Varda ROTTER, Rarda (IL); Naomi GOLDFINGER, Rehovot (IL); Shalom MADAR, Rehovot (IL); Yan STEIN, Rehovot (IL)

(21) Appl. No.: 14/766,151

(22) PCT Filed: Feb. 19, 2014
(86) PCT No.: PCT/IL2014/050178

§ 371 Date: Aug. 6, 2015

Related U.S. Application Data
(60) Provisional application No. 61/766,714, filed on Feb. 20, 2013.

(51) Int. Cl. A61K 38/21; C12Q 1/68; G01N 33/574; C07K 14/565; C07K 14/56

(52) U.S. Cl. CPC A61K 38/215; C07K 14/565; C07K 14/56; A61K 38/212; G01N 33/57496; C12Q 1/6886; G01N 2333/4748; C12Q 2600/158; C12Q 2600/106; C12Q 2600/156

(57) ABSTRACT

The present invention is directed to cancer therapy, specifically to prognostic and therapeutic compositions and methods, wherein expression of a mutated form of p53 serves as a predictive marker for successful type I interferon (IFN) treatment. The invention also relates to the use of low doses of IFN having reduced clinical toxicity, and enables the use of IFN to prevent tumor formation in susceptible patient populations such as patients carrying p53 germline mutations.

### FIELD OF THE INVENTION

The present invention is directed to cancer therapy, specifically to improved type I interferon treatment having enhanced efficacy and reduced toxicity.

### BACKGROUND OF THE INVENTION

Neoplasia is a process in which the normal controlling mechanisms that regulate cell growth and differentiation are impaired, resulting in progressive growth and tumor formation.

This impairment of control mechanisms allows the tumor to enlarge and occupy spaces in vital areas of the body. If the tumor invades surrounding tissue and is transported to distant sites (metastases) it will likely result in death of the individual.

The desired goal of cancer therapy is to eliminate cancer cells preferentially, without having a deleterious effect on normal cells. Several methods have been used in an attempt to reach this goal, including surgery, radiation therapy and chemotherapy. However, more effective therapies with fewer side effects are clearly needed.

p53, a well-known tumor suppressor, is frequently mutated in tumors resulting in the expression of tumor promoting mutant forms. Several studies have addressed the role of mutant p53 in the tumor-stroma interaction (Solomon et al., 2011). For example, mutant p53 expressed in stromal cells surrounding prostate tumors, enhances tumor growth and facilitates metastasis.

In addition, a clear correlation was revealed between mutant p53 and VEGF expression, and tumor aggressiveness. Mutant p53 was also reported to cooperate with E2F to induce the expression of ID4, which in turn leads to augmented angiogenesis.

Mutant p53 is also implicated in the etiology and pathology of Li-Fraumeni syndrome, also known as the Sarcoma, Breast, Leukemia and Adrenal Gland (SBLA) syndrome. The syndrome is linked to germline mutations of p53, which can be inherited or can arise de novo early in embryogenesis or in one of the parents' germ cells. Because the p53 gene is responsible for initiating DNA repair mechanisms and/or apoptosis upon detection of DNA damage, Li-Fraumeni syndrome, with one of the two p53 copies already mutated, predisposes a person to cancer development because only one additional mutation (in the second p53 allele) is necessary to impair a significant portion of the tumor suppressor system. Indeed, persons with this syndrome have an approximately 25-fold increased risk of developing a malignant tumor by age 50 than the population average, and are at risk for a wide range of malignancies, with particularly high occurrences of breast cancer, brain tumors, acute leukemia, soft tissue sarcomas, bone sarcomas, and adrenal cortical carcinoma. Currently, effective prophylactic measures for Li-Fraumeni syndrome are lacking, and patient advice mainly includes comprehensive annual physical examination for early tumor detection.

Vast efforts have been made world-wide to target mutant p53 in cancer and offer a tailor made solution for individuals carrying p53 mutations. Drugs such as low-molecular weight compounds and short peptides were developed, aimed at restoring p53 wild-type activity, by shifting the wild-type and mutant equilibrium towards the wild-type conformation. Such are the compound MIRA-1 (Bykov et al., 2005) and the short peptides CDB3 and CP-31398 (Foster et al., 1999; Samuels-Lev et al., 2001). However, none of these drugs have been approved for clinical use.

Interferons (IFNs) are a group of cytokines that serve as a defense mechanism against viral infections and have the capacity to affect the transformation process. There are two major types of interferons—type I IFNs, mainly represented by IFN-α and IFN-β, and type II IFN, represented by IFNγ. Type I IFNs are produced by all nucleated cells, they bind a cell surface receptor encoded by Interferon (alpha, beta and omega) Receptor 1 (IFNAR1/2) and can potentially initiate four different pathways. The canonical pathway includes the activation of JAK1 and TYK1, which relays the signal onto STAT1/2 by phosphorylation. STAT1/2 form a complex with IRF9 that translocates to the nucleus, where it binds IFN-stimulated response elements (ISRE) residing in the promoters of IFN target genes. IFN-β seems to have a pleiotropic effect on cancer. On the one hand, IFN-β directly inhibits tumor growth when secreted by the tumor microenvironment (Studeny et al., 2004). On the other hand, IFN-β partakes in tumor escape from the immune system, either by selecting for IFN non-responsive cells (Dunn et al., 2006) or by contributing to oncogenic Ras transformation (Tsai et al., 2011) and enriching for cancer initiating cells (Tjandra et al., 2007). Although IFN-β seems to cooperate with wild type p53 in tumor suppression and stress responses (Chiantore et al., 2012; Takaoka et al., 2003), its interaction with the mutant forms of p53 has not been elucidated.

Several publications reported that IFNs induce apoptosis in certain tumor cells. For example, Porta et al. (2005) disclose that in MCF-7 and SKNSH cell lines, IFN-α and IFN-γ induce p53-dependent and -independent apoptosis, respectively. Notably, Porta et al. further disclose that type I IFN enhances the transcription of p53. Juang et al. (2004) disclose that various human colon cancer cell lines tested in vitro differed significantly in susceptibility to growth inhibition or apoptosis induced by recombinant human IFN-β (rHuIFN-β). Two p53-mutant lines, COH and CC-M2, derived from high-grade colon adenocarcinoma, showed signs of apoptosis after treatment with 250 IU/ml of rHuIFN-β in the culture medium. The similarly p53-mutated HT-29 line from a grade I adenocarcinoma showed no apoptosis, however, and only cell cycle G1/G0 or S phase retardation upon incubation with a significantly higher concentration, namely 1000 IU/ml rHuIFN-β. The effects of IFNs on tumor cell migration or p53 levels were not examined, and no guidance for evaluating the responsiveness of tumor cells to IFN therapy is provided.

Noguchi et al. (2008) report that IFN-α and β inhibit neovascularization and induce apoptosis in hepatocellular carcinoma (HCC) tumors; IFN-β, but not IFN-α, inhibited HCC growth in a mouse model, at doses of 10⁴ IU twice a week. These effects were shown to be independent of p53 status, as HCC apoptosis and tumor size in the mouse model were comparable in HCC cells expressing wild type and mutant p53 (BNL or PLC cells, respectively).

Yuan et al. report that IFN-α increased doxorubicin-induced cytotoxicity to a much greater degree through apoptosis in human osteosarcoma p53-wild U2OS cells, but not p53-mutant MG63 cells.

IFNs have been approved for a number of indications, including hairy cell leukemia, chronic myelogenous leukemia, malignant melanoma, follicular lymphoma, condylomata acuminata, AIDs-related Kaposi sarcoma, and chronic hepatitis B and C (IFN-α2), multiple sclerosis (IFN-β) and chronic granulomatous disease and malignant osteopetrosis (IFN-γ). These proteins or derivatives thereof are further being evaluated as potential therapeutic agents for certain other indications. For example, U.S. Pat. No. 8,158,129 discloses a method of treating cancer or a viral infection with a dimeric PEGylated IFN-α or IFN-β complex defined by certain amino acid sequences. Gene therapy methods wherein vectors expressing IFN proteins are implemented in cancer therapy have been suggested e.g. in U.S. Pat. No. 7,256,181 and U.S. Pat. Appl. Pub No. 2003/229042. The use of IFNs as a supplemental therapy for augmenting or modulating other cancer treatments has further been contemplated e.g. in U.S. Pat. No. 6,096,301 and U.S. Pat. Appl. Pub Nos. 2012/082647 and 2008/089860. Several clinical trials test the use of IFN-β for a number of additional indications, including as viral therapy in treating patient with liver cancer, for treating patients with metastatic cutaneous melanoma or ocular melanoma, as an adenoviral-based gene therapy vector for certain malignancies, and in combination therapy with AZT in patients with AIDS-related Kaposi's sarcoma.

However, despite their potential therapeutic value, IFN proteins have only had limited clinical success; the therapeutic outcome of IFN treatment in a particular individual is currently unpredictable. For example, the response rate of IFN-α against HBV and HCV is only approximately 20%, and the majority of clinical trials in which IFN are used in the treatment of solid tumors (either alone or in combination with conventional chemotherapeutic agents) have been unsuccessful. In addition, the promise of IFN therapy e.g. as a cancer therapeutic has been hindered due to its considerable systemic toxicity. Most studies have shown that the tumoricidal effect of IFN was due to direct cytotoxicity against the tumor cells at relatively high doses, which are unacceptable for certain patient populations. In the clinical practice, continuous administration of high doses of IFN resulted in several symptoms including fatigue, anorexia, weight loss, dizziness, and severe hematological disorders.

There remains an unmet medical need for providing improved cancer therapy, having enhanced therapeutic efficacy and/or reduced toxicity. Specifically, means for identifying patient populations amenable for effective IFN treatment, as well as means for reducing IFN-associated side effects, are of need. Therapeutic modalities for the treatment and prevention of cancer and other pathologies associated with mutant p53 expression, such as Li-Fraumeni syndrome, are further required.

### SUMMARY OF THE INVENTION

The present invention is directed to cancer therapy, specifically to prognostic and therapeutic compositions and methods, wherein expression of a mutated form of p53 serves as a predictive marker for successful type I interferon (IFN) treatment. According to particular embodiments, the invention relates to the use of low doses of IFN having reduced clinical toxicity, and enables the use of IFN to prevent tumor formation in susceptible patient populations, such as patients carrying p53 germline mutations.

The invention is based, in part, on the surprising discovery that type I IFNs exert a destabilizing effect that is specific to oncogenic mutant p53, while the levels of wild-type p53 tumor suppressor remain intact. It is herein disclosed unexpectedly that upon encounter with cancer cells, Cancer Associated Fibroblasts (CAFs) activate the IFN-β pathway, however when mutant p53 is present in the cancer cells, this pathway is less pronounced. Addition of exogenous IFN-β was surprisingly found to overcome the suppressive activity of mutant p53, and reduce its RNA levels and protein levels. In addition, administering IFN-β to mutant p53 bearing cancer cells exerted an inhibitory effect on their capacity to migrate which corresponds to metastatic ability.

Thus, it is unexpectedly disclosed, that IFN-β is particularly effective in the treatment of tumors expressing a mutated form of p53, compared to tumors expressing wild-type p53 or lacking p53 expression altogether. Accordingly, the invention relates in some embodiments to diagnostic assays for determining whether a subject is amenable for cancer treatment with a type I IFN, by determining whether the subject expresses mutant p53. The invention further relates to therapeutic methods, comprising identifying a subject afflicted with cancer as expressing mutant p53, and if the subject expresses mutant p53, administering a type I IFN to said subject.

IFNs were surprisingly found to exert a novel biological function that is unrelated to their previously reported functions such as apoptosis induction, namely specifically reducing mutant p53 levels. Thus, the invention now provides therapeutic use of type I IFN at low doses that are ineffective or insufficient to induce apoptosis, thereby minimizing adverse effects associated with IFN treatment. Accordingly, the invention provides for improved IFN therapy, providing both enhanced efficacy and improved safety.

In addition, since the use of low doses of type I IFN is contemplated, providing enhanced safety under prolonged chronic treatment, the invention now enables for the first time prophylactic use of type I IFN in preventing tumor formation or tumor spreading, including in subjects susceptible to, but not afflicted with, a neoplasm. Thus, for example, the invention for the first time enables the use of IFNs in the treatment of disorders associated with germline p53 mutations, such as Li-Fraumeni syndrome. According to some embodiments the compositions and methods of the invention as described herein are particularly useful for prevention of metastases due to the activity which limits cancer cell migration.

Thus, according to a first aspect of the present invention, there is provided a method comprising the steps of:
a) determining whether a subject expresses mutant p53; and
b) if the subject expresses mutant p53, identifying said subject as amenable for treatment with a type I IFN.

According to another embodiment, if said subject is identified as being amenable for treatment with a type I IFN, the method further comprises the step of:
c) administering an effective amount of a type I IFN to said subject identified as being amenable for treatment with a type I IFN.

As used herein, a subject that “expresses mutant p53” or the term “mutant p53 expression”, refer to expression by at least some cells (e.g. tumor cells) of a subject, of a p53 protein that is structurally different from wild type p53 protein, due to one or more mutation in the p53 gene. The structural difference in mutant p53 proteins typically results in loss of some or all of the tumor suppressor functions of this protein, and many times to gain of oncogenic properties. Mutant p53 proteins may result from point mutations or deletion mutations in the p53 gene. A variety of different p53 mutations have been identified which are associated with a number of different malignancies. See Hollstein, M. et al. (1991) Science, 253:49-53, for a description of p53 mutations found in number of different cancers. This term is further meant to differentiate said subject from subjects expressing only p53 protein having the native wild-type conformation, as well as from p53-null subjects, that do not substantially express the p53 protein.

In another aspect, the invention provides a method for determining whether a subject is amenable for treatment with a type I IFN, comprising the steps of:
a) determining whether the subject expresses mutant p53 characterized by one or more mutations residing within the DNA binding domain; and
b) if the subject expresses the mutant p53, identifying said subject as amenable for treatment with the type I IFN.

In another embodiment, said treatment with said type I IFN is used for reducing cellular levels of mutant p53.

In one embodiment, the type I IFN is an IFN-β or an IFN-α.

In another embodiment, determining whether the subject expresses mutant p53 is performed by determining the presence of mutant p53 protein or of mutant p53 mRNA transcript.

Advantageously, the invention now discloses for the first time that type I IFNs such as IFN-β specifically reduce the levels of mutant p53 mRNA. This is particularly surprising in view of the teachings of the art (Porta et al., Supra), indicating that type I IFN enhances the transcription of p53. In another embodiment, determining whether a subject expresses mutant p53 is performed by determining the presence of mutant p53 mRNA, wherein if said subject expresses mutant p53 mRNA, said subject is determined to be amenable for treatment with the type I IFN. In another embodiment, determining whether the subject expresses mutant p53 further comprises determining mutant p53 mRNA levels, wherein if said subject expresses mutant p53 mRNA, and the level of the mutant p53 mRNA is reduced upon type I IFN treatment, said subject is determined to be amenable for treatment with the type I IFN. In another embodiment said mutant p53 is characterized by one or more mutations that down-regulate the level of said p53 mRNA upon exposure to type I IFN.

In another embodiment the subject is afflicted with or is predisposed to developing a neoplastic disorder. In another embodiment said disorder is Li-Fraumeni syndrome or another disorder associated with a p53 germline mutation. In another embodiment the disorder is cancer, cancer metastasis, or another disorder characterized by mutation in the p53 gene (e.g somatic mutation). In another embodiment said subject is a cancer patient in a state of remission. In another embodiment said subject is predisposed to developing the disorder.

In another embodiment, if said subject is identified as being amenable for treatment with the type I IFN, the method further comprises the step of:
c) administering an effective amount of said type I IFN to said subject identified as being amenable for treatment with said type I IFN.

In another embodiment, the type I IFN is IFN-β. In another embodiment, the type I IFN is IFN-α. In another embodiment said method is used for the treatment of cancer. In another embodiment said method is used for inhibition of cancer metastasis. In another embodiment said method is used for treating a subject afflicted with a disorder associated with a p53 mutation. In another embodiment said disorder is Li-Fraumeni syndrome or another disorder associated with a p53 germline mutation. In another embodiment said method is used for sensitizing a chemotherapy-resistant tumor to chemotherapy. In another embodiment said method is used for reducing cellular levels of mutant p53, thereby treating cancer or metastasis in said subject. In another embodiment said method is used for reducing cellular levels of mutant p53, thereby treating a subject afflicted with a disorder associated with a p53 mutation. In another embodiment the disorder is Li-Fraumeni syndrome. In another embodiment said method is used for reducing cellular levels of mutant p53, thereby sensitizing a chemotherapy-resistant tumor to chemotherapy.

In another embodiment, said type I IFN is administered to said subject at low doses that ensure efficacy while minimizing side effects associated with the induction of apoptosis. In another embodiment said IFN is IFN-β1. In another embodiment said IFN is administered to said subject as a sole active ingredient.

In another embodiment, if said subject is identified as being amenable for treatment with the type I IFN, the method further comprises the step of:
c) contacting cells of said subject with said type I IFN.

In another embodiment, step c) is performed ex vivo.

In another embodiment, if said subject is identified as being amenable for treatment with the type I IFN, the method further comprises the step of:
c) expressing in cells of said subject said type I IFN.

In another embodiment step c) is performed ex vivo.

In another aspect, there is provided a kit for identifying a subject as being amenable for treatment with a type I IFN, comprising:
a) means for determining whether the subject expresses mutant p53; and
b) instructions for identifying said subject as amenable for treatment with a type I IFN if said subject expresses mutant p53.

In another embodiment said IFN is IFN-β. In another embodiment, the type I IFN is IFN-α.

In another aspect the invention provides a pharmaceutical pack, comprising:
a) a therapeutically effective dose of a type I IFN; and
b) instructions for administering said type I IFN to a subject identified as expressing mutant p53.

In another embodiment, the subject is afflicted with or is predisposed to developing a neoplastic disorder. In another embodiment, the disorder is cancer and/or metastases and/or is characterized by mutation in the p53 gene. In another embodiment said disorder is Li-Fraumeni syndrome. In another embodiment said therapeutically effective dose ensures efficacy while minimizing side effects associated with the induction of apoptosis. In another embodiment said IFN is IFN-β. In another embodiment, the type I IFN is IFN-α.

In another aspect the invention is directed to a type I IFN for use in the treatment or prevention of a neoplastic disorder, in a subject identified as expressing mutant p53 that is characterized by one or more mutations residing within the DNA binding domain.

In another embodiment, determining whether the subject expresses mutant p53 is performed by determining the presence of mutant p53 protein or of mutant p53 mRNA transcript. In another embodiment, determining whether the subject expresses mutant p53 is performed by determining the presence of mutant p53 mRNA, wherein if said subject expresses mutant p53 mRNA, said subject is determined to be amenable for treatment with the type I IFN.

In another embodiment, determining whether the subject expresses mutant p53 further comprises determining mutant p53 mRNA levels, wherein if said subject expresses mutant p53 mRNA, and the level of the mutant p53 mRNA is reduced upon type I IFN treatment, said subject is determined to be amenable for treatment with said type I IFN.

In another embodiment the subject is afflicted with or is predisposed to developing the neoplastic disorder. In another embodiment said disorder is Li-Fraumeni syndrome or another disorder associated with a p53 germline mutation. In another embodiment the disorder is cancer or cancer metastasis. In another embodiment said subject is a cancer patient in a state of remission. In another embodiment said subject is predisposed to developing the disorder.

In another embodiment said type I IFN is formulated for in vivo administration. In another embodiment said type I IFN is formulated for ex vivo administration.

In another embodiment said type I IFN is used for reducing cellular levels of mutant p53, thereby treating said subject. In another embodiment said type I IFN is used for reducing cellular levels of mutant p53, thereby sensitizing a chemotherapy-resistant tumor to chemotherapy, treating or preventing cancer or metastasis, or treating Li-Fraumeni syndrome in said subject.

In another embodiment said type I IFN is formulated as a pharmaceutical composition in unit dosage form, at a dose that does not substantially induce apoptotic cell death when administered to said subject.

In another embodiment said IFN is an IFN-β or an IFN-α. In another embodiment said IFN is IFN-β, preferably IFN-β1. In another embodiment said IFN is IFN-α, preferably IFN-α2.

In another embodiment said type I IFN is used as a sole active ingredient.

In another aspect the invention provides a nucleic acid construct for use in the treatment or prevention of a neoplastic disorder,

wherein:
the construct comprises a nucleic acid sequence encoding a type I IFN, which nucleic acid sequence is operably linked to a transcription-regulating sequence, and
the subject is identified as expressing mutant p53 that is characterized by one or more mutations residing within the DNA binding domain.

Other objects, features and advantages of the present invention will become clear from the following description and drawings.

### BRIEF DESCRIPTION OF THE DRAWINGS

FIG. 1. An in vitro model to study the tumor-stroma encounter in lung cancer. (A). p53-null lung carcinoma cells (H1299) were introduced with the designated mutations. p53 levels were determined by Western blot analysis (A) and by QRT-PCR (B).

FIG. 2. The IFN beta pathway is up regulated in fibroblasts after co-cultivation with mutp53-bearing carcinoma cells. (A) To assess the significance of the physical interaction between IIK3-T and II1299¹⁷⁵ in eliciting the IFN pathway, IIK3-T were introduced to conditioned media from each cell type grown alone or from a co-culture plate. mRNA levels of two known IFN targets were measured by QRT-PCR. (B) IFN α and β mRNA levels. (C) CAFs and H1299 were grown either alone or in a co-culture. The co-cultured cells were incubated with the designated antibodies. Shown is the mRNA level of the designated IFN targets. *P<0.05.

FIG. 3. Expression patterns of H1299 in response to co-cultivation with CAFs. (A) A heat-map depicting genes that exhibit Pearson correlation of at least 0.9 to representative bait(s) for “responsiveness” (top), “over-induction” (middle) and “attenuation” (bottom) expression patterns. Beneath is the percentage of IFN targets in each list based on the ‘Interferome database’. (B) QRT-PCR analysis of a representative gene from each expression pattern. (C-E) The H1299 panel was treated with the designated IFNs for 24 h. Shown is a QRT-PCR analysis of MX1 expression

FIG. 4. Mutant p53 counteracts IFN-β by SOCS1-mediated attenuation of STAT1 phosphorylation. (A) Cells were treated with IFN-β for 16 h, fixed and sorted by an “Image stream” FACS. The upper panel depicts representative images from each condition and the graph represents the mean pixel intensity of STAT1 positive cells for the entire population. (B) Same as in A, here the graph shows the similarity between p-STAT and DAPI staining, thereby quantifying both the expression and localization of pSTAT1. (C) Cells were treated with IFN-β for the designated durations, shown is a graph depicting nuclear p-STAT1. (D) The cells were also collected for RNA analysis and a QRT-PCR for SOCS1 expression was performed. (E) H1299¹⁷⁵ cells were introduced with RNAi against LacZ as a control or against SOCS1. (F) Cells were then treated with IFN-β for 24 h. Shown is a QRT-PCR analysis of MX1 expression. p<0.05. (G) Cells were seeded in trans-wells in serum-free media and treated with IFN-β for 24 h. Migrating cells were collected and counted.

FIG. 5. IFN-β reduces mutant p53 mRNA levels by inhibiting its RNA stabilizer WIG1 (ZMAT3). Cells were treated with IFN-β for the designated time points, fixed and sorted by an “Image stream” FACS. (A) Representative images from each condition and (B) a graph representing the mean pixel intensity of mutant p53 positive cells for the entire population. (C) Cells were treated with the designated IFNs for 24 h and mutant p53 and GAPDH levels were measured by western blot. Cells were treated with IFN-β for 24 h and mutant p53 (D) and WIG1 (E) RNA levels were determined by QRT-PCR. *P<0.05. (F) HepG2 cells were introduced with mut175 plasmid and treated with IFN-β. RNA levels were determined by QRT-PCR. *P<0.05.

FIG. 6. A schematic representation of the interaction between mutant p53 and the IFN-β pathway. Blue arrows denote positive effect while red arrows denote negative effect. Upon encounter with cancer cell CAFs activate the IFN-β pathway which limits cancer cells' migration. When mutant p53 is present in the cancer cells, this pathway is moderated via SOCS1 mediated inhibition of STAT1 phosphorylation. IFN-β is able to reduce mutant p53 RNA levels by attenuating the expression of mutant p53 RNA stabilizer WIG1.

FIG. 7. MX1 is attenuated by mutant p53 following IFN-β treatment. (A) A549 were introduced with an empty vector or mutant p53 (R175H) vector, and treated with IFN-β for 24 h. Shown is a QRT-PCR for MX1. (B) SKBR3 which express endogenous mutant p53 (R175H) were introduced with shRNA vector targeting non-human sequence and mutant p53 and treated with IFN-β for 24 h. Shown is a QRT-PCR for MX1.

FIG. 8. SOCS1 mediates mutant p53 attenuating effect on CXCL11 following IFN-β treatment. H1299¹⁷⁵ cells were introduced with RNAi against LacZ as a control or SOCS1 and treated with IFN-β for 24 h. Shown is QRT-PCR for SOCS1 and MX1 expression.

FIG. 9. IFN-β attenuates WIG1 in mouse B-cells. (A) mouse B cells were treated with IFN-β and gene expression was determined using a microarray. Shown is the average WIG1 expression of four replicates. p=0.003

FIG. 10. YNS, an IFN-α analog, reduces mutant p53 levels. (A+B). H1299^{control} and H1299¹⁷⁵ sublines were treated with YNS or IFN-β for the designated time points. Shown are p53 mRNA levels measured by qPCR (A) and protein levels measured by western blot (B). (C+D). PC-3 prostate cancer sublines, which are null for p53 or exogenously overexpressing different p53 mutated alleles, were treated by YNS or IFN-β for 24 hours. Shown are p53 mRNA levels measured by qRT-PCR (C), protein levels that were measured by western blot (D) and quantified by densitometry analysis (E).

### DETAILED DESCRIPTION OF THE INVENTION

The present invention is directed to cancer therapy, specifically to diagnostic and therapeutic compositions and methods, wherein expression of a mutated form of p53 serves as a predictive marker for successful type I interferon (IFN) treatment, such as IFN-β treatment. By pre-determining the subject population that will respond to IFN treatment, enhanced safety may be obtained, by 1) preventing unwanted side effects in patients in which the clinical benefits of the treatment are limited, and 2) avoiding toxicity resulting from excessive IFN doses, used in an attempt to ensure a therapeutic benefit. According to particular embodiments, the invention relates to the use of low doses of IFN having reduced clinical toxicity, and enables the use of IFN to prevent tumor formation in susceptible patient populations such as patients carrying p53 germline mutations.

The invention is based, in part, on the surprising discovery that addition of exogenous IFN-β overcomes the suppressive activity of mutant p53 on IFN-β secretion by CAFs, and reduces mutant p53 levels (both RNA and protein levels). Without wishing to be bound by a single theory or mechanism of action, this effect was found to be mediated by attenuating the expression of mutant p53 RNA stabilizer WIG1. In addition, administering IFN-β to mutant p53 bearing cancer cells, exerted an inhibitory effect on their capacity to migrate, which corresponds to metastatic ability. It is further surprisingly shown by the invention that IFN-α is also able to decrease mutant p53 levels.

Thus, according to a first aspect of the present invention, there is provided a method comprising the steps of:
a) determining whether a subject expresses mutant p53; and
b) if the subject expresses mutant p53, identifying said subject as amenable for treatment with a type I IFN.

According to another embodiment, if said subject is identified as being amenable for treatment with a type I IFN, the method further comprises the step of:
c) administering an effective amount of a type I IFN to said subject identified as being amenable for treatment with a type I IFN.

While the invention conveniently relates to in vivo administration of IFN, other ex vivo methods may be contemplated. Thus in another embodiment, if said subject is identified as being amenable for treatment with a type I IFN, the method further comprises the step of:
c) contacting (or expressing in) cells of said subject a type I IFN.

According to various embodiments, said contacting may be performed in vivo or ex vivo (e.g. in the treatment of hematologic malignancies, wherein tumor-containing blood cells are isolated from the subject and incubated with IFN, and thereafter administered back to the patient). According to other embodiments, nucleic acid vehicles may be used to express a type I IFN in cells of subjects expressing mutant p53, according to recombinant methods and protocols known in the art.

In another embodiment, the method is used for determining whether a subject in need thereof is amenable for treatment with a type I IFN, and comprises the steps of:
a) determining whether the subject expresses mutant p53 characterized by one or more mutations residing within the DNA binding domain; and
b) if the subject expresses the mutant p53, identifying said subject as amenable for treatment with the type I IFN.

In another aspect the invention is directed to a type I IFN for use in the treatment or prevention of a neoplastic disorder, in a subject identified as expressing mutant p53 that is characterized by one or more mutations residing within the DNA binding domain.

In another aspect the invention provides a nucleic acid construct for use in the treatment or prevention of a neoplastic disorder,

wherein:
the construct comprises a nucleic acid sequence encoding a type I IFN, which nucleic acid sequence is operably linked to a transcription-regulating sequence, and
the subject is identified as expressing mutant p53 that is characterized by one or more mutations residing within the DNA binding domain.

According to certain embodiments, the type I IFN is an IFN-β or an IFN-α. In a particular embodiment, said IFN is IFN-β. In various other embodiments, the invention contemplates the use of isolated IFN, recombinant IFN, or an IFN derivative, modified e.g. by PEGylation or other half-life elongating moieties.

In certain embodiments, the mutant p53 is characterized by one or more mutations residing within the DNA binding domain (e.g. R175H or R248Q). In other embodiments, determining whether the subject expresses mutant p53 may be performed by a variety of methods as known in the art. For example, determining the level (or presence) of p53 protein may be determined e.g. using Western blotting, enzyme-linked immunosorbent assay (ELISA) or immunohistochemistry (e.g. on tumor or other tissue biopsies) using antibodies specific to the mutant p53 conformation. Determining the level (or presence) of the p53 mRNA transcript may be performed e.g. by reverse transcriptase-polymerase chain reaction (RT-PCR) or Northern blotting. Non limiting exemplary protocols for affecting these assays are described in the Examples below.

Advantageously, the invention now discloses for the first time that IFN-β specifically reduces the levels of mutant p53 mRNA. This is particularly surprising in view of the teachings of the art (Porta et al., Supra), indicating that type I IFN enhances the transcription of p53. Thus, advantageous embodiments of the present invention relate to determining whether a subject expresses mutant p53 by determining mutant p53 mRNA levels. In a specific embodiment, the invention provides for determining that a subject is amenable for treatment with a type I IFN if said subject expresses mutant p53 mRNA. In another specific embodiment, the invention provides for determining that a subject is amenable for treatment with a type I IFN if said subject expresses mutant p53 mRNA, and the level of the mutant p53 mRNA is reduced upon type I IFN treatment. For example, this may conveniently be performed by isolating tumor cells from said subject and incubating these cells e.g. in vitro in the presence of a type I IFN (e.g. 0.001-1 nM or 0.1-1 nM IFN-β for 12-48 hrs), wherein if the level of the mutant p53 mRNA is decreased (a statistically significant difference or a difference recognized by a person of skill in the art), identifying said subject as amenable for treatment with a type I IFN.

In another embodiment, the subject is afflicted with a neoplastic disorder. In another embodiment the subject is predisposed to (has an enhanced risk to developing, but is not afflicted with) a neoplastic disorder. In another embodiment, the disorder is cancer. In a particular embodiment, said cancer is a solid tumor. In another embodiment, the disorder is characterized by mutation in the p53 gene. In another embodiment, the mutation is a p53 germline mutation. In a particular embodiment, said disorder is Li-Fraumeni syndrome. In another embodiment said subject is a cancer patient in a state of remission (not afflicted with a clinically detectable tumor). In another embodiment, the disorder is cancer metastasis.

Administering an effective amount of a type I IFN to said subject identified as being amenable for treatment with a type I IFN according to the teachings of the invention may be used for several therapeutic purposes. In another embodiment, the method is used for the treatment of cancer. In another embodiment, the method is used for the prevention of appearance of cancer. In another embodiment, the method is used for the prevention of spreading of cancer. In another embodiment the method is used for prevention or inhibition of cancer metastasis. In another embodiment the method is used for treating a subject afflicted with a disorder associated with a p53 mutation, e.g. a disorder characterized by a p53 germline mutation. In a particular embodiment, the disorder is Li-Fraumeni syndrome. In another embodiment, the method is used for sensitizing a chemotherapy-resistant tumor to chemotherapy.

In another embodiment the method is used for reducing cellular levels of mutant p53. In another embodiment the method is used for reducing cellular levels of mutant p53, thereby treating or preventing cancer or metastasis in said subject. In another embodiment the method is used for reducing cellular levels of mutant p53, thereby treating a subject afflicted with a disorder associated with a p53 mutation, e.g. a disorder characterized by a p53 germline mutation. In a particular embodiment, the disorder is Li-Fraumeni syndrome. In another embodiment, the method is used for reducing cellular levels of mutant p53, thereby sensitizing a chemotherapy-resistant tumor to chemotherapy.

Advantageously, the present invention contemplates the therapeutic use of type I IFN at low doses that are sub-optimal for inducing apoptotic cell death or that do not substantially induce apoptosis. In another embodiment, type I IFN is administered to said subject at low doses that ensure efficacy while minimizing side effects associated with the induction of apoptosis. In another embodiment, type I IFN is administered to said subject at low doses that ensure efficacy while minimizing side effects associated with type I IFN therapy. In another embodiment, minimizing is reducing the risk of any side effect or a combination of side effects within a treated population by at least 10%, 20%, 40%, 50%, 60%, 75%, 80%, or 90%. In another embodiment, minimizing is reducing the frequency of any side effect or a combination of side effects in a patient by at least 10%, 20%, 40%, 50%, 60%, 75%, 80%, or 90%. In certain other embodiments, said type I IFN is administered in an amount that reduces its ability to cause apoptotic cell death by at least 10%, 20%, 40%, 50%, 60%, 75%, 80%, or 90%.

According to some embodiments, said IFN is administered to said subject as a sole active ingredient.

In another aspect, the invention relates to a kit for identifying a subject as being amenable for treatment with a type I IFN, comprising:
a) means for determining whether the subject expresses mutant p53; and
b) instructions for identifying said subject as amenable for treatment with a type I IFN if said subject expresses mutant p53.

In a particular embodiment, said IFN is IFN-β.

In another aspect, there is provided pharmaceutical pack, comprising:
a) a therapeutically effective dose of a type I IFN; and
b) instructions for administering said type I IFN to a subject identified as expressing mutant p53.

According to some embodiments, the subject is afflicted with or is predisposed to developing a neoplastic disorder, e.g. cancer and/or metastases and/or a disorder characterized by mutation in the p53 gene. In a particular embodiment said disorder is Li-Fraumeni syndrome.

In another embodiment said type I IFN is administered to said subject at low doses that ensure efficacy while minimizing side effects associated with the induction of apoptosis. In another embodiment, said IFN is IFN-β.

Type I Interferon

Type I IFN polypeptides are well known and characterized in the art, having common structural and functional properties. Structurally, type I IFNs are class II α-helical cytokines sharing marked sequence homology, a shared three-dimensional core structure, and common chromosomal locations (Levy et al., 2011, Dunn et al., 2006), and known type I IFN sequences may be found in GenBank. Functionally, a type I IFN is characterized in that it specifically binds a cell surface receptor encoded by Interferon (alpha, beta and omega) Receptor 1 (IFNAR1/2), thereby inducing signaling pathways mediated by downstream effectors of the IFNAR1/2 receptor in an agonist manner. Accordingly, a type I IFN referred to in the compositions and methods of the invention is a type I IFN polypeptide as known in the art (either naturally occurring or synthetic) i.e. that is recognized by a skilled artisan as a type I IFN, based on its structural and functional properties, as described herein.

IFNAR1/2 is a heterodimeric receptor composed of two chains, IFNAR1 and IFNAR2. This receptor signals through two Janus family kinases, Tyk2 and Jak1, resulting in recruitment of STAT1 to receptor-bound STAT2 and formation of STAT1-STAT2 heterodimers that dissociate from the receptors and migrate into the nucleus.

Preferably, the type I IFN is a mammalian type I IFN. The mammalian types are designated IFN-α (alpha), IFN-β (beta), and further include the less abundantly prevalent IFN-κ (kappa), IFN-δ (delta), IFN-ε (epsilon), IFN-τ (tau), IFN-ω (omega), and IFN-ζ (zeta, also known as limitin), present in certain species.

Human type I IFNs number 13 distinct non-allelic α proteins, one β, and one ω. Each member is a mature protein of 165 or 166 amino acid residues, all sharing the same structure, having 2 conserved disulfide bonds, Cys1-Cys98 and Cys29-Cys138, and binding the same cell surface receptor, IFNAR1/2.

The IFN-α proteins are produced by leukocytes. They are mainly involved in innate immune response against viral infection. The 13 IFN-α subtypes are encoded by IFNA1, IFNA2, IFNA4, IFNA5, IFNA6, IFNA7, IFNA8, IFNA10, IFNA13, IFNA14, IFNA16, IFNA17, IFNA21, found together in a cluster on chromosome 9. Synthetic IFN-α are also available commercially (e.g. pegylated IFN alfa-2a, pegylated IFN alfa-2b). The IFN-β proteins are produced in large quantities by fibroblasts. They are known to have antiviral activity that is involved mainly in innate immune response. IFN-β polypeptides include IFN-β1 (IFNB1) and IFN-β3 (IFNB3). Synthetic IFN-β include, for example, human IFN-β1A, produced in mammalian cells (AVONEX), and IFN-β1B, produced in modified E. coli (EXTAVIA, BETASERON).

According to certain embodiments, the type I IFN referred to in the compositions and methods of the invention is an IFN-β or an IFN-α (including naturally occurring IFN-β or an IFN-α and isoforms or variants thereof, and analogs and chemical derivatives thereof known in the art). Preferably, said IFN is IFN-β. In a particular embodiment, said IFN is IFN-β, e.g. IFN-β1 or IFN-β3. In another specific embodiment, said IFN-β is an IFN-β1. In another specific embodiment, said IFN-β is an IFN-β3. In another embodiment said IFN is IFN-α, e.g. IFN-α1 and IFN-α2. In a particular embodiment said IFN-α is an IFN-α2. In another particular embodiment said IFN-α is an IFN-α2B.

In certain other particular embodiments, the type I IFN used in the compositions and methods of the invention is a human type I IFN, e.g. human IFN-β or human IFN-α. According to certain preferable embodiments, the use of human IFN-β is contemplated. In another specific embodiment, said IFN-β is human IFN-β1. In another specific embodiment, said IFN-β is human IFN-β3.

For example, without limitation, the IFN-β may have the following amino acid sequence:

(mature human IFN-β1, SEQ ID NO: 1)
MSYNLLGFLQ RSSNFQCQKL LWQLNGRLEY CLKDRMNFDI
PEEIKQLQQF QKEDAALTIY EMLQNIFAIF RQDSSSTGWN
ETIVENLLAN VYHQINHLKT VLEEKLEKED FTRGKLMSSL
HLKRYYGRIL HYLKAKEYSH CAWTIVRVEI LRNFYFINRL
TGYLRN.

In another embodiment, the human type I IFN is human IFN-α2.

For example, without limitation, the IFN-α may have the following amino acid sequence:

(mature human IFN-α2, SEQ ID NO: 40)
CDLPQTHSLG SRRTLMLLAQ MRRISLFSCL KDRHDFGFPQ
EEFGNQFQKA ETIPVLHEMI QQIFNLFSTK DSSAAWDETL
LDKFYTELYQ QLNDLEACVI QGVGVTETPL MKEDSILAVR
KYFQRITLYL KEKKYSPCAW EVVRAEIMRS FSLSTNLQES
LRSKE.

In various other embodiments, the invention contemplates the use of isolated IFN, recombinant IFN, or an IFN derivative, modified e.g. by PEGylation or other half-life elongating moieties, wherein each possibility represents a separate embodiment of the invention.

In another embodiment, the type I IFN is conjugated to a half life elongating moiety. In a particular embodiment, the IFN is an IFN-β, conjugated to a half life elongating moiety. For example, U.S. Pat. Nos. 8,557,232 and 7,670,595 disclose IFN-β derivatives, stabilized by fusion of an immunoglobulin Fc region, and U.S. Pat. No. 7,338,788 discloses additional IFN-β variants and conjugates. In a particular embodiment, the IFN is an IFN-α, conjugated to a half life elongating moiety.

Recombinant and/or synthetic methods for producing such IFN proteins are known in the art. Conveniently, various commercially available IFNs may be used in the compositions and method of the invention, including IFN analogs and derivatives characterized by improved activity. The doses of such analogs and derivatives may be adjusted by the skilled artisan based on their relative in vivo activity (e.g. reported pharmacokinetic and pharmacodynamic data). It is to be understood, that such analogs and derivatives retain high structural (sequence) similarity to the corresponding native IFN polypeptide sequence, typically at least 90% homology, preferably at least 95% homology, such that they are recognized by the skilled artisan to be structural equivalents. These analogs and derivatives are characterized by affinity to IFNAR1/2 that is at least equivalent to the native type I IFN, thus providing signaling via IFNAR that mediates a biological activity of a type I IFN in vivo, at least to a degree sufficient to exert therapeutic activity (e.g. to cancer).

For example, BETASERON marketed by BAYER is a recombinant human IFN-β, in which cysteine 17 is substituted with serine, thus preventing incorrect disulfide bond formation. Such analogs, wherein the cysteine at position 17 is deleted or replaced by a neutral amino acid are also described in U.S. Pat. No. 4,588,585. U.S. Patent Application Publication No. 2004/0230040 discloses IFN-α2 variants with added cysteine residues.

WO 2007/000769 and U.S. Pat. No. 7,919,078, incorporated herein by reference, disclose recombinant IFNα2 polypeptides comprising certain amino acid substitutions compared to the native human IFNα2, residing within amino acid residues 57-89 and/or 159-165 (numbering relating to the mature human IFNα2 polypeptide, i.e. to SEQ ID NO: 40 as set forth herein). Specifically, IFNα2 polypeptides having an improved agonist activity contain gain-of-function mutation(s) in residues 57, 58, and/or 61. More specifically, these polypeptides are characterized by substitution of the histidine at position 57 (H57) to a residue selected from the group consisting of tyrosine and methionine; (b) substitution of the glutamate at position 58 (E58) to a residue selected from the group consisting of asparagine, aspartate, leucine, and alanine; and (c) substitution of the glutamine at position 61 (Q61) to a residue selected from the group consisting of serine, leucine, and aspartate; and combinations thereof. These mutations are said to increase the affinity of the resulting human IFNα2 polypeptide for the IFNR1 subunit of the IFNR1/2 relative to wild type IFNα2, maintaining or even enhancing their agonist activity.

These modified IFNs may further contain additional mutations, e.g. at the C′ end. Specifically, a negative or neutral residue among the 7 carboxy-terminal residues of the IFNα2 protein may be substituted by a residue selected from lysine and arginine. For example, the IFNα2 polypeptides may further comprise an additional substitution, aside from the aforementioned substitution at residues H57, E58 and/or Q61, selected from the group consisting of (a) the glutamate at position 159 (E159), (b) the serine at position 160 (S160), (c) the leucine at position 161 (L161), (d) the serine at position 163 (S163), and (e) the glutamate at position 165 (E165) to a residue selected from lysine and arginine. Thus, the IFNα2 polypeptides may further comprises an additional substitution of the 5-10 C-terminal residues of said IFNβ2 polypeptide to a sequence selected from the group consisting of KRLKSKE (SEQ ID NO: 41) and KRLKSK (SEQ ID NO: 42).

Preferred polypeptides disclosed by WO 2007/000769 and U.S. Pat. No.7,919,078 comprise at least two and preferably three of the mutations (amino acid substitutions) at positions 57, 58 and 61. Exemplary IFNβ2 polypeptides disclosed by these publications include e.g. polypeptides comprising a triple mutant selected from the group consisting of: H57A, E58A, Q61A; H57M, E58D, Q61L; and H57Y, E58N, Q61S. Optionally, these polypeptides comprise the C-terminal substitutions as indicated above. The suggested dose for these polypeptides as disclosed therein is from about 10 μg to about 100 μg for administration to a 70 kg person.

The present invention discloses unexpectedly, that a recombinant IFNa2 polypeptide in accordance with the disclosure of WO 2007/000769 and U.S. Pat. No. 7,919,078, was able to specifically reduce the levels of mutant p53 in lung cancer and prostate cancer cell lines. Thus, according to some embodiments, the type I IFN used in the compositions and methods of the invention is a human IFNα2 characterized by substitution of at least one, preferably at least two and most preferably three amino acids at positions 57, 58, and 61, and optionally C-terminal substitutions, as described herein (yet maintaining the structural and functional properties of type I IFNs as detailed herein and at least 90% amino acid homology to human IFNα2).

Thus, according to certain embodiments, the type I IFN is an IFN-α2 that differs from native IFN-α2 by at least two substitutions, selected from the group consisting of: (a) substitution of the histidine at position 57 (H57) to a residue selected from the group consisting of tyrosine and methionine; (b) substitution of the glutamate at position 58 (E58) to a residue selected from the group consisting of asparagine, aspartate, leucine, and alanine; (c) substitution of the glutamine at position 61 (Q61) to a residue selected from the group consisting of serine, leucine, and aspartate; and (d) substitution of the 5-10 C-terminal residues of said IFN-α2 to a sequence selected from the group consisting of KRLKSKE (SEQ ID NO: 41) and KRLKSK (SEQ ID NO: 42). In other words, the polypeptides according to this embodiment differ from native IFN-α2 by the substitutions described in (a)-(e), and are otherwise identical to the respective native (naturally occurring) IFN-α2.

According to particular embodiments, the IFNα2 polypeptide comprises at least two of the substitutions H57Y, E58N, and Q61S. In another particular embodiment, the IFNα2 polypeptide comprises both of the substitutions H57Y and Q61S. In another particular embodiment, the IFNα2 polypeptide comprises two substitutions selected from H57Y, E58N, and Q61 S. In another particular embodiment, the IFNα2 polypeptide comprises all three substitutions H57Y, E58N, and Q61S in native human IFNα2. In another particular embodiment, the IFNα2 polypeptide comprises all three substitutions H57Y, E58N, and Q61S in native human IFNα2, and further comprises a substitution of the C terminal ESLRSKE to KRLKSKE (SEQ ID NO: 41). Preferably, the IFNα2 polypeptide exhibits improved IFNAR agonist activity, relative to native human IFNα2 (SEQ ID NO: 40).

According to other particular embodiments, the IFNα2 polypeptide comprises at least two of the substitutions H57M, E58D, and Q61L. In another embodiment, the IFNα2 polypeptide comprises both of the substitutions H57Y and Q61L. In another embodiment, the IFNα2 polypeptide comprises two substitutions selected from H57M, E58D, and Q61L. In another embodiment, the IFNα2 polypeptide comprises all three of the substitutions H57M, E58D, and Q61L in native human IFNα2. In another embodiment, the IFNα2 polypeptide comprises all three of the substitutions H57M, E58D, and Q61L in native human IFNα2, and further comprises a substitution of the C terminal ESLRSKE to KRLKSKE (SEQ ID NO: 41). Preferably, the IFNα2 polypeptide exhibits improved IFNAR agonist activity, relative to native human IFNα2.

According to yet other particular embodiments, the IFNα2 polypeptide comprises at least two of the substitutions H57Y, E58L, and Q61D. In another embodiment, the IFNα2 polypeptide comprises both of the substitutions H57Y and Q61D. In another embodiment, the IFNα2 polypeptide comprises two substitutions selected from H57Y, E58L, and Q61D. In another embodiment, the IFNα2 polypeptide comprises all three of the substitutions H57Y, E58L, and Q61D in native human IFNα2. In another embodiment, the IFNα2 polypeptide comprises all three of the substitutions H57Y, E58L, and Q61D in native human IFNα2 and further comprises a substitution of the C terminal ESLRSKE to KRLKSKE (SEQ ID NO: 41). Preferably, the IFNα2 polypeptide exhibits improved IFNAR agonist activity, relative to native human IFNα2.

According to further particular embodiments, the IFNα2 polypeptide comprises at least two of the substitutions H57Y, E58A, and Q61S. In another embodiment, the IFNα2 polypeptide comprises two substitutions selected from H57Y, E58A, and Q61S. In another embodiment, the IFNα2 polypeptide comprises all three of the substitutions H57Y, E58A, and Q61S in native human IFNα2. In another embodiment, the IFNα2 polypeptide comprises all three of the substitutions H57Y, E58A, and Q61S in native human IFNα2 and further comprises a substitution of the C terminal ESLRSKE to KRLKSKE (SEQ ID NO: 41). Preferably, the IFNα2 polypeptide exhibits improved IFNAR agonist activity, relative to native human IFNα2.

According to additional particular embodiments, the IFNα2 polypeptide comprises at least two of the substitutions H57A, E58A, and Q61A. In another embodiment, the IFNα2 polypeptide comprises two substitutions selected from H57A, E58A, and Q61A. In another embodiment, the IFNα2 polypeptide comprises all three of the substitutions H57A, E58A, and Q61A in native human IFNα2. In another embodiment, the IFNα2 polypeptide comprises all three of the substitutions H57A, E58A, and Q61A in native human IFNα2 and further comprises a substitution of the C terminal ESLRSKE to KRLKSKE (SEQ ID NO: 41). Preferably, the IFNα2 polypeptide exhibits improved IFNAR agonist activity, relative to native human IFNα2.

P53

p53 is a tumor suppressor protein, which acts as a major barrier against cancer progression. p53 responds to various types of cellular stress, and triggers cell cycle arrest, apoptosis, or senescence. This is achieved by transcriptional transactivation of specific target genes carrying p53 DNA binding motifs. Most of the p53 mutations identified in cancer patients are missense point mutations that target the DNA-binding core domain (DBD) of p53, thereby abolishing specific DNA binding of p53 to its target site. This prevents p53-dependent transcription and consequently p53-mediated tumor suppression.

Structural studies have revealed that the tumor-derived missense mutations in the DBD of p53 produce a common effect: destabilization of DBD folding at physiological temperature. This destabilization may be reversible, since some mutants can revert to wild-type conformation and bind DNA at reduced temperature. Thus, most mutations of p53 destabilize p53 protein folding, causing partial denaturation at physiological temperature (37° C.).

p53 can assume either a wild type, active conformation or a mutant, misfolded inactive conformation. The two conformational states of p53 can be distinguished, for example, by two specific monoclonal antibodies, PAb240 and PAb1620. PAb240 binds to residues 212-217 in the DBD of p53. This region is inaccessible to the antibody (Ab) in the wild type (wt) conformation. However, in denatured or mutant p53, it is exposed. PAb1620 recognizes a conformational, nonlinear epitope in the DBD, composed of two distinct regions of p53 and including residues R156, L206, R209 and N210. In the wt conformation the protein is folded in a way that holds the loops in close proximity to each other, forming the complete epitope recognized by PAb1620. When p53 protein is misfolded (as a result of mutation, temperature, denaturation or the like), these two loops move farther away, the epitope is destroyed and therefore the mutant conformation is PAb1620 negative (Wang et al., 2001).

As used herein, the terms “wild-type p53” and “wt p53” may interchangeably be used and are directed to a p53 protein having the structure and conformation of a wt p53 protein at physiological temperature and hence, activity of a wt p53 protein, as described herein. Conveniently, wt p53 can be identified by a specific monoclonal antibody, recognizing the wt p53 conformation.

The term “conformation” with respect to a protein is directed to the three-dimensional structural arrangement (folding) of a protein in space.

As used herein, the terms “mutant p53”, “mutp53”, “mutated p53”, and “p53 mutant” may interchangeably be used and are directed to a p53 protein in which a structural difference from wt p53, due to one or more mutation in the p53 gene, results in impairment in functional activities, as described herein. The structural difference in mutant p53 proteins is typically manifested by a misfolded conformation at physiological temperature, resulting in impaired activity. The impaired activity may typically contain reduction or loss of the ability to bind a target site, e.g. a DNA binding region or a downstream protein effector. These changes result in loss of some or all of the tumor suppressor functions of this protein and/or gain of oncogenic properties. Thus, it is to be understood that “silent” mutations (such as conservative substitution point mutations) that do not affect the conformation and activity of the protein, are excluded from this definition.

In certain embodiments, the mutant p53 is characterized by one or more mutations residing within the DNA binding domain (DBD, e.g. R175H or R248Q). In some embodiments, a mutp53 is mutated at the DBD region. In some embodiments, a mutp53 is misfolded in an inactive conformation. In some embodiments, a mutp53 is misfolded in a conformation characterized by a reduction of at least 50%, and preferably at least 60%, 70%, 80% or 90% in DNA binding activity. In some embodiments, a mutp53 is mutated at the DBD region and is misfolded in an inactive conformation. In some embodiments, a mutp53 is mutated at the DBD region and is misfolded in a conformation characterized by a reduction of at least 50%, and preferably at least 60%, 70%, 80% or 90% in DNA binding activity. In some exemplary embodiments, the mutp53 is a temperature sensitive (ts) mut p53 R249S (wherein the arginine at position 249 is substituted by serine, R249Sp53), a hot spot full length mutant p53 R175H (R175H), or an R248Q mutation. In another embodiment the mutp53 is characterized by one or more missense point mutations that target the DBD of p53, thereby abolishing specific DNA binding of p53 to its target site. In some embodiments, a mutp53 is identified by a specific monoclonal antibody, e.g. an antibody capable of recognizing a misfolded conformation of p53 (induced by the p53 mutation).

The structure of the p53 gene and protein has been extensively studied, and hence defined structural domains corresponding to specific functions are known (and readily identified by one of skill in the art). These include the N-terminal transactivation domains, the central DBD, and the C-terminal regulatory domains.

For example, human p53 can be divided into several domains, including:

The amino-terminus part that comprises the acidic transactivation domain, containing the Highly Conserved Domain I (HCD I; residing at amino acids 1-42), and further the mdm2 protein binding site (minimal binding domain at residues 18-26). The following segment (residues 40-92) contains a series of repeated proline residues that are conserved in the majority of p53. It also contains a second transactivation domain. The central region (amino acids 101-306) contains the DNA binding domain (DBD). It is the target of most p53 mutations found in human cancers. It contains HCD II to V. The oligomerization domain (residues 307-355) consists of a beta-strand, followed by an alpha-helix necessary for dimerization, as p53 is composed of a dimer of two dimers. A nuclear export signal (NES) is localized in this oligomerization domain. The carboxy-terminus of p53 (residues 356-393) contains 3 nuclear localization signals (NLS) and a non-specific DNA binding domain that binds to damaged DNA. This region is also involved in downregulation of DNA binding of the central domain. The amino acid positions indicated above are relative to the human p53 of SEQ ID NO: 2 (see Example 5 below).

Subjects and Disorders

In another embodiment, the subject is afflicted with a neoplastic disorder. In another embodiment the subject is predisposed to (has an enhanced risk to developing, but is not afflicted with) a neoplastic disorder. In another embodiment, the disorder is cancer.

For example a cancer can include, without limitation an adrenocortical carcinoma, anal cancer, bladder cancer, brain tumor, glioma, breast carcinoma, carcinoid tumor, cervical cancer, colon carcinoma, endometrial cancer, esophageal cancer, extrahepatic bile duct cancer, Ewings tumor, extracranial germ cell tumor, eye cancer, gall bladder cancer, gastric cancer, germ cell tumor, gestational trophoblastic tumor, head and neck cancer, hypopharyngeal cancer, islet cell carcinoma, kidney cancer, laryngeal cancer, leukemia, lip and oral cavity cancer, liver cancer, lung cancer, lymphoma, melanoma, mesothelioma, merkel cell carcinoma, metastatic squamous head and neck cancer, myeloma, nasopharyngeal cancer, neuroblastoma, oral cancer, oropharyngeal cancer, osteosarcoma, ovarian cancer, pancreatic cancer, sinus and nasal cancer, parathyroid cancer, penile cancer, pheochromocytoma cancer, pituitary cancer, plasma cell neoplasm, prostate cancer, rhabdomyosarcoma, rectal cancer, renal cell carcinoma, salivary gland cancer, skin cancer, Kaposi's sarcoma, T-cell lymphoma, soft tissue sarcoma, stomach cancer, testicular cancer, thymoma, thyroid cancer, urethral cancer, uterine cancer, vaginal cancer, vulvar cancer, or Wilms' tumor, wherein each possibility represents a separate embodiment of the invention.

In a particular embodiment, the cancer is a solid tumor. For example, the solid tumor (cancer) may be an adrenocortical carcinoma, anal cancer, bladder cancer, brain tumor, glioma, breast carcinoma, cervical cancer, colon carcinoma, endometrial cancer, esophageal cancer, extrahepatic bile duct cancer, Ewings tumor, extracranial germ cell tumor, eye cancer, gall bladder cancer, gastric cancer, germ cell tumor, gestational trophoblastic tumor, head and neck cancer, hypopharyngeal cancer, islet cell carcinoma, kidney cancer, laryngeal cancer, lip and oral cavity cancer, liver cancer, lung cancer, melanoma, mesothelioma, merkel cell carcinoma, metastatic squamous head and neck cancer, myeloma, nasopharyngeal cancer, neuroblastoma, oral cancer, oropharyngeal cancer, osteosarcoma, ovarian cancer, pancreatic cancer, sinus and nasal cancer, parathyroid cancer, penile cancer, pituitary cancer, prostate cancer, rhabdomyosarcoma, rectal cancer, renal cell carcinoma, salivary gland cancer, skin cancer, soft tissue sarcoma, stomach cancer, testicular cancer, thyroid cancer, urethral cancer, uterine cancer, vaginal cancer, vulvar cancer, or Wilms' tumor, wherein each possibility represents a separate embodiment of the invention.

According to further particular embodiments, the cancer is a solid tumor selected from the group consisting of liver cancer (e.g. hepatocellular carcinoma), lung cancer (e.g. lung carcinoma such as non-small cell lung carcinoma and lung adenocarcinoma), breast cancer (e.g. breast carcinoma such as breast adenocarcinoma), and colon cancer (e.g. colorectal carcinoma). Each possibility represents a separate embodiment of the invention.

In another embodiment, the cancer is a carcinoma (e.g. hepatocellular, lung, breast or colorectal carcinoma). In another embodiment, the cancer is adenocarcinoma. In another embodiment, the cancer is hepatocellular carcinoma. In another embodiment the cancer is lung carcinoma (e.g. small cell lung carcinoma, non-small cell lung carcinoma, or adenocarcinoma).

In another embodiment the cancer is breast carcinoma. In another embodiment the cancer is colorectal carcinoma. In another embodiment, the cancer is sarcoma.

In another embodiment, the disorder is characterized by mutation in the p53 gene. In another embodiment, the mutation is a p53 somatic mutation. In another embodiment, the mutation is a p53 germline mutation. In a particular embodiment, said disorder (characterized by the p53 germline mutation) is Li-Fraumeni syndrome (LFS).

A definition of LFS originated from Li and Fraumeni's work as a proband with a sarcoma aged under 45 years with a first-degree relative aged under 45 years with any cancer, plus an additional first- or second-degree relative in the same lineage with any cancer aged under 45 years or a sarcoma at any age. Birch et al. subsequently formulated a definition for Li-Fraumeni-like syndrome (LFL) based on more extensive and updated information of the types of tumors and the ages of onset in families. LFL is defined as a proband with any childhood tumor, or a sarcoma, brain tumor, or adrenocortical tumor aged under 45 years plus a first- or second-degree relative in the same lineage with a typical LFS tumor at any age, and an additional first- or second-degree relative in the same lineage with any cancer under the age of 60 years. In the past, the diagnosis of LFS and LFL was made by clinical criteria, i.e. based on the signs and symptoms the patient and family had. Now, genetic testing is available to identify p53 germline mutations, which underline the majority of LFS and a portion of LFL patients, thus enabling diagnosis of these conditions before any physical signs of LFS appear.

In another embodiment said subject is a cancer patient in a state of remission (not afflicted with a clinically detectable tumor). In another embodiment, the disorder is cancer metastasis. According to particular embodiments, the methods of the invention may be used for the prevention of cancer metastasis, in a subject not afflicted with a clinically detectable tumor.

According to embodiments of the invention, type I IFN is used for the treatment of subjects expressing mutant p53, as the expression and pathological activity of mutant p53 have been surprisingly found to be reduced by type I IFN treatment. Accordingly, various embodiments of the invention are directed to methods for determining amenability to type I IFN treatment in subjects afflicted with or predisposed to various p53-dependent disorders, that are characterized by (and have an etiology and/or pathology associated with) mutant p53 expression. Thus, it is to be understood that subjects that are already receiving a type I IFN for the treatment of a disorder that is p53-independent are excluded from these methods. In another embodiment, the subject is not concurrently afflicted with MS, viral hepatitis (e.g. chronic hepatitis B or C), or other viral infections for which type I IFN treatment is indicated.

Therapeutic Use

Administering an effective amount of a type I IFN (or otherwise providing a type I IFN therapy as detailed herein) to said subject identified as being amenable for treatment with a type I IFN according to the teachings of the invention may be used for several therapeutic purposes. In another embodiment, the method is used for the treatment of cancer. In another embodiment, the method is used for the prevention of appearance of cancer. In another embodiment, the method is used for the prevention of spreading of cancer. In another embodiment the method is used for prevention or inhibition of cancer metastasis. In another embodiment the method is used for treating a subject afflicted with a disorder associated with a p53 mutation, e.g. a disorder characterized by a p53 germline mutation. In a particular embodiment, the disorder is Li-Fraumeni syndrome. In another embodiment, the method is used for sensitizing a chemotherapy-resistant tumor to chemotherapy.

In another embodiment the method is used for reducing cellular levels of mutant p53. In another embodiment the method is used for reducing cellular levels of mutant p53, thereby treating or preventing cancer or metastasis in said subject. In another embodiment the method is used for reducing cellular levels of mutant p53, thereby treating a subject afflicted with a disorder associated with a p53 mutation, e.g. a disorder characterized by a p53 germline mutation. In a particular embodiment, the disorder is Li-Fraumeni syndrome. In another embodiment, the method is used for reducing cellular levels of mutant p53, thereby sensitizing a chemotherapy-resistant tumor to chemotherapy. In another embodiment, the methods are used of treating or inhibiting the spread of micrometastases.

Typically, the type I IFN is formulated in the form of a pharmaceutical composition, which may further comprise one or more pharmaceutically acceptable carriers, excipients and/or diluents. The compositions described herein can be administered e.g. by a parenteral mode (e.g., subcutaneous, intraperitoneal, or intramuscular injection). The phrases “parenteral administration” and “administered parenterally” as used herein mean modes of administration other than enteral and topical administration, usually by injection, and include, subcutaneous or intramuscular administration, as well as intravenous, intracapsular, intraorbital, intracardiac, intradermal, intraperitoneal, transtracheal, subcuticular, subcapsular, subarachnoid, intraspinal, epidural, and intrasternal injection and infusion. Each possibility represents a separate embodiment of the invention. Administration of the dose can be intravenous, subcutaneous, intramuscular, or any other acceptable systemic method. In a particular embodiment a type I IFN of the invention is administered subcutaneously. In another particular embodiment a type I IFN of the invention is administered intravenously. In another particular embodiment a type I IFN of the invention is administered intramuscularly. Based on the judgment of the attending clinician, the amount of drug administered and the treatment regimen used will, of course, be dependent on the age, sex, and medical history of the patient being treated, the neutrophil count, the severity of the specific disease condition, and the tolerance of the patient to the treatment as evidenced by local toxicity and by systemic side-effects. According to certain other embodiments, other modes of administration, such as oral administration and transdermal administration (e.g. using a transdermal patch) are contemplated. In another embodiment, continuous administration e.g. via a subcutaneous pump (e.g. Medtronic MiniMed® pump) may be employed.

Advantageously, the present invention contemplates the therapeutic use of type I IFN at low doses that are sub-optimal for inducing apoptotic cell death or that do not substantially induce apoptosis. In another embodiment, type I IFN is administered to said subject at low doses that ensure efficacy while minimizing side effects associated with the induction of apoptosis. In another embodiment, type I IFN is administered to said subject at low doses that ensure efficacy while minimizing side effects associated with type I IFN therapy. In another embodiment, minimizing is reducing the risk of any side effect or a combination of side effects within a treated population by at least 10%, 20%, 40%, 50%, 60%, 75%, 80%, or 90%. In another embodiment, minimizing is reducing the frequency of any side effect or a combination of side effects in a patient by at least 10%, 20%, 40%, 50%, 60%, 75%, 80%, or 90%. In certain other embodiments, said type I IFN is administered in an amount that reduces its ability to causes apoptotic cell death by at least 10%, 20%, 40%, 50%, 60%, 75%, 80%, or 90%.

For example, IFN-β is currently indicated for the treatment of multiple sclerosis at a recommended dose of 30 micrograms once a week (IFN β-1A, AVONEX), or at 0.25 mg injected subcutaneously every other day (IFN β-1B, EXTAVIA). IFN-α is typically used at doses higher than those of IFN-β, as known in the art. For example, IFN-α is indicated for treatment of chronic myelogenous leukemia at a dose of 9 MIU daily administered as a subcutaneous injection (IFN α-2a, ROFERON-A). Other doses of type I IFN have been reported to induce apoptosis in cancer cells (see e.g. Noguchi et al. 2008, reporting the use of type I IFN in cell culture and in animal models). In addition, the ED50 of recombinant human IFN-β of SEQ ID NO:1 on apoptosis of HeLa cells has been reported to be 20-30 ng/ml. According to some embodiments, the invention contemplates the use of type I IFNs at doses substantially lower than are currently used, thereby minimizing side effects in the subject in need thereof.

A “substantially lower” dose is a statistically significant reduced amount, not hitherto considered by those of skilled in the art (such as the treating physicians) to be an effective treatment for cancer. Thus, low doses that ensure efficacy while minimizing side effects associated with the induction of apoptosis according to the present invention, are doses that are lower by at least a power of ten, preferably lower by two powers of ten and most preferably lower by at least three powers of ten, compared to the currently used doses.

Preferably, the use of low doses of IFN-β is contemplated. For example, low doses for in vivo administration (e.g. to a human subject) may be e.g. 1 ng-1 μg, for instance 1 ng-100 ng, 10 ng-100 ng, 1 ng-10 ng or 10 ng-1 μg IFN-β (e.g. recombinant human IFN-β1). Low doses of IFN-β for ex vivo use may be e.g. 0.001-0.01 nM.

In some embodiments, the present invention herein demonstrates that type I IFN provides in mutant p53-expressing subject an enhanced anti-neoplastic effect that is sufficient for effective use in the absence of other anti-cancer agents. Thus, according to some embodiments, the methods of the invention contemplate the use of type I IFN and in particular IFN-β as a sole active ingredient.

Yet, in other embodiments, the invention relates to the use of that type I IFN in combination (concurrent or sequential) with other drugs, such as chemotherapeutic drugs or other anti-cancer agents known in the art, e.g. when used for sensitizing a subject (having a chemotherapy-resistant tumor) to chemotherapy. For example, the type I IFN may be administered in combination with e.g. a chemotherapy such as taxol and/or cisplatin or an immunotherapy such as an antibody directed to a tumor associated antigen. In another embodiment, the chemotherapeutic drug is taxol, methotrexate, mitomycin C, bleomycin, 5-Fluorouracil, novantrone, carbo and cis platinum, adriamycin, vincristine or combinations thereof.

In another embodiment, the compositions and methods of the invention encompass administration of a combination of type I IFNs (e.g. concurrently or sequentially administering an IFN-α and an IFN-β. In another embodiment, the type I IFN may be administered in combination with a type II IFN (e.g. a human IFN-γ).

In some embodiments, the invention relates to methods in which, if a subject is identified as being amenable for treatment with a type I IFN as described herein, the method further comprises contacting cells of said subject with a type I IFN.

According to some embodiments, contacting cells of said subject with a type I IFN may be performed in vivo, by administering an effective amount of a type I IFN to said subject identified as being amenable for treatment with a type I IFN. Conveniently, the type I IFN may be administered to the subject in the form of a pharmaceutical composition which may further comprise one or more pharmaceutically acceptable carriers, excipients and/or diluents, as detailed herein.

An “effective amount” (or “therapeutically effective dose”) refers to that amount of a type I IFN which, when administered to a subject in need thereof (i.e. identified as amenable for treatment with a type I IFN, in accordance with the teachings of the invention), is sufficient to effect treatment of the condition or disorder. In the methods of the invention, a subject in need thereof may be treated with a dose of e.g. 1 ng-0.5 mg IFN-β, preferably a low dose of 1 ng-1 μg IFN-β (for example IFN-β1, e.g. human IFN-β1), which may be administered e.g. s.c. once a day, or every 2, 3, 5 or 7 days, as long as a therapeutic benefit is obtained. A subject in need thereof may be treated in other embodiments with a dose of e.g. 10 ng-50 mg IFN-α (e.g. human IFN-α2), preferably a low dose of 10 ng-10 μg or 10 ng-1 μg IFN-α, which may be administered e.g. s.c. once a day, or every 2, 3, 5 or 7 days, as long as a therapeutic benefit is obtained.

In other embodiments, contacting cells of said subject with a type I IFN may be performed ex vivo. Conveniently, these embodiments may be performed by obtaining cells from said subject, incubating the cells ex vivo in the presence of an effective amount of a type I IFN, and re-introducing the cells to said subject. A non-limitative example for applying these methods is in the treatment of hematologic malignancies, wherein tumor-containing blood cells are isolated from the subject, using methods well-known in the art, and contacted (incubated) with the respective IFN (e.g. 0.001-1nM or 0.1-1 nM IFN-β for 12-48 hrs), and thereafter administered back to the subject.

Methods for obtaining and isolating cells from cell-containing samples and for collection of appropriate cell samples are known in the art. For example, whole blood samples may be collected from the subject of interest into a suitable anticoagulant (e.g. ethylenediaminetetraacetate (EDTA) or acid citrate dextrose), and cells of interest may be separated or isolated from whole blood using methods commonly used in the art, e.g. by one or more techniques including filtration, density fractionation (e.g. centrifugation of whole blood, centrifugation of blood in multiple stages, and continuous-flow centrifugation), or by using antibody-based methods to deplete unwanted blood components.

Advantageously, cells of said subject may be incubated in some embodiments with a type I IFN at low concentrations that do not substantially induce apoptosis, e.g. 0.001-0.01 nM IFN-β (e.g. IFN-β1).

Kits, Assays and Pharmaceutical Packs

According to some embodiments, the invention relates to methods comprising determining whether a subject expresses mutant p53.

In certain embodiments, the mutant p53 is characterized by one or more mutations residing within the DNA binding domain (e.g. R175H or R248Q). In other embodiments, determining whether the subject expresses mutant p53 may be performed by a variety of methods as known in the art. For example, determining the level of p53 protein may be performed by various immunoassays e.g. using Western blotting, enzyme-linked immunosorbent assay (ELISA) or immunohistochemistry (e.g. on tumor or other tissue biopsies), as detailed herein. Conveniently, antibodies specific to the mutant p53 conformation may be used in these immunoassays. Determining the level of the p53 mRNA transcript may be performed by various nuclic acid-based assays, e.g. by RT-PCR or Northern blotting, as detailed herein. Non limiting protocols for affecting these assays are demonstrated in the Examples herein, and will be discussed further below.

According to other embodiments the present invention relates to determining whether a subject expresses mutant p53 by determining mutant p53 mRNA levels (e.g. the presence or absence of mutant p53 mRNA). In a specific embodiment, the invention provides for determining that a subject is amenable for treatment with a type I IFN if said subject expresses mutant p53 mRNA. In another specific embodiment, the invention provides for determining that a subject is amenable for treatment with a type I IFN if said subject expresses mutant p53 mRNA, and the level of the mutant p53 mRNA is reduced upon type I IFN treatment (e.g. ex vivo contacting). For example, this may conveniently be performed by isolating tumor cells from said subject and incubating these cells e.g. in vitro in the presence of a type I IFN (e.g. 0.001-1 nM or 0.1-1 nM IFN-β for 12-48 hrs), wherein if the level of the mutant p53 mRNA is reduced (decreased) with a statistically significant difference (or a difference recognized by a person of skill in the art), identifying said subject as amenable for treatment with a type I IFN.

According to various embodiments of the invention, determining whether a subject expresses mutant p53 is affected by obtaining a cell containing sample from the subject (e.g. a tissue sample such as a tumor biopsy or a fluid sample, e.g. blood or urine sample or a specimen containing fluids that were in contact with the primary tumor site), and assaying the presence of mutant p53 (e.g. protein or mRNA) in said sample, e.g. by any of the methods herein disclosed. Such samples may be collected and optionally processed prior to assaying (e.g. to enrich for cells in the sample or to isolate mRNA) as known in the art. In various embodiments, positive control samples (e.g. from cells expressing mutant p53) and negative control samples (e.g. from cells expressing native p53 or not expressing p53) may conveniently be used as reference in these bioassays, as known in the art.

In some embodiments, the methods of the invention are non-invasive methods, wherein the sample is obtained non-invasively (e.g. the sample is a secreted sample, obtained from urine, saliva or other bodily secretions), and the methods are not performed inside a human body (e.g. ex vivo).

Thus, according to some embodiments, there are provided ex vivo methods for determining whether a subject in need thereof is amenable for treatment with a type I IFN, comprising the steps of:
a) determining (i.e. ex vivo) whether the subject expresses mutant p53, wherein the mutant p53 is characterized by one or more mutations residing within the DNA binding domain; and
b) if the subject expresses said mutant p53, identifying said subject as amenable for treatment with the type I IFN.

In another aspect, the invention relates to a kit for identifying a subject as being amenable for treatment with a type I IFN, comprising:
a) means for determining whether the subject expresses mutant p53; and
b) instructions for identifying said subject as amenable for treatment with a type I IFN if said subject expresses mutant p53.

In a particular embodiment, said IFN is IFN-β.

In another aspect, there is provided pharmaceutical pack, comprising:
a) a therapeutically effective dose of a type I IFN; and
b) instructions for administering said type I IFN to a subject identified as expressing mutant p53.

According to some embodiments, the subject is afflicted with or is predisposed to developing a neoplastic disorder, e.g. cancer and/or metastases and/or a disorder characterized by mutation in the p53 gene. In a particular embodiment said disorder is Li-Fraumeni syndrome.

In another embodiment said type I IFN is administered to said subject at low doses that ensure efficacy while minimizing side effects associated with the induction of apoptosis. In another embodiment, said IFN is IFN-β. Thus, the instructions may comprise instructions for administering said type I IFN to a subject identified as expressing mutant p53 at low doses that ensure efficacy while minimizing side effects associated with the induction of apoptosis (e.g. at the low doses as specified herein).

According to some embodiments, means for determining whether the subject expresses mutant p53 may include, for example, reagents for detecting mutant p53 polypeptide, or in other embodiments, reagents for detecting mutant p53 mRNA.

For example, without limitation, mutant p53 polypeptide may be detected by various immunoassays such as Western blotting, ELISA or immunohistochemistry using antibodies (or other binding agents) specific to the mutant p53 conformation. Thus, the means may contain one or more p53-specific antibodies as described herein, and optionally one or more control antibodies. Such antibodies are known in the art.

Exemplary specific binding agents (antibodies) for use in immunoasays in the methods and kits of the inventiom include e.g. monoclonal antibodies, polyclonal antibodies, and antibody fragments such as recombinant antibody fragments, single-chain antibodies (scFv) and the like. Single-chain antibodies are small recognition units consisting of the variable regions of the immunoglobulin heavy (V_{H}) and light (V_{L}) chains which are connected by a synthetic linker sequence.

For example, PAb240, described supra, is specific to the mutant p53 conformation, while PAb1 620 (described supra) and PAB246 are specific for the wild-type conformation (Wang et al., 2001). Other antibodies recognize both the wild-type and mutant p53, such as PAb421 that is specific for the carboxy terminal domain of p53 conserved in both wild-type and mutant p53 (Arai et al., 1986). Since the epitope and antigen-binding regions sequences of these antibodies have been characterized and are known in the art, the use of recombinant or humanized antibodies, or antibody fragments containing the antigen-binding regions of these antibodies, as well as antibodies and fragments that specifically recognize their corresponding epitopes, may be readily synthesized and employed in the methods and kits of the invention.

Methods of generating monoclonal and polyclonal antibodies are well known in the art. Antibodies may be generated via any one of several known methods, which may employ induction of in vivo production of antibody molecules, screening of immunoglobulin libraries, or generation of monoclonal antibody molecules by continuous cell lines in culture. Antibody fragments may be obtained using methods well known in the art, including, but not limited to by proteolytic hydrolysis of the antibody or by expression in E. coli or mammalian cells (e.g., Chinese hamster ovary (CHO)) cell culture or other protein expression systems) of DNA encoding the fragment. Single-chain Fvs are prepared by constructing a structural gene comprising DNA sequences encoding the heavy chain variable and light chain variable domains connected by an oligonucleotide encoding a peptide linker. The structural gene is inserted into an expression vector, which is subsequently introduced into a host cell such as E. coli. The recombinant host cells synthesize a single polypeptide chain with a linker peptide bridging the two variable domains.

In another embodiment, detecting mutant p53 polypeptide may be performed using an immunoassay such as an ELISA testing kit. In such assays, for example, media samples from each vessel are typically incubated in the presence of an immobilized first specific binding agent (e.g. an antibody) capable of specifically binding p53 (e.g. mutant p53). Binding of p53 to the first specific binding agent may be measured using any one of a variety of known methods, such as using a labeled second specific binding agent capable of specifically binding p53 (at a different epitope). In some embodiments, various conventional tags or labels may be used, such as a radioisotope, an enzyme, a chromophore or a fluorophore. A typical radioisotope is iodine-125 or sulfur-35. Typical enzymes for this purpose include horseradish peroxidase, horseradish galactosidase and alkaline phosphatase.

Alternately, other immunoassays may be used; such techniques are well known to the ordinarily skilled artisan and have been described in many standard immunology manuals and texts.

In some embodiments, the methods of the invention are suitable for automated or semi-automated analysis, and may enable clinical, medium or high-throughput screening of multiple samples. For example, automated ELISA systems such as Biotest's Quickstep® ELISA Processor, Maxmat Automated microwell ELISA analyzer (Maxmat S. A., France), or DSX™ Four-Plate System (Dynex Technologies) may conveniently be used.

Other suitable assays include for example flow cytometry assays (such as singleplex and multiplex bead-based Luminex® assays (Invitrogen).

Alternately, an antibody microarray may be used. The antibody microarray comprises antibodies directed to the polypeptide of interest, e.g. anti-p53 antibodies. In general, the sample obtained from the subject is placed on the active surface of a chip for a sufficient time to allow binding. Then, unbound molecules are washed from the surface using a suitable eluant, such as phosphate buffered saline. In general, the more stringent the eluant, the more tightly the polypeptide of interest must be bound to be retained after the wash. The retained polypeptide of interest now can be detected by appropriate means.

Additional exemplary assays may be based on dipstick technology, as demonstrated, for example, in U.S. Pat. Nos. 4,632,901, 4,313,734 and 4,786,589 5,656,448 and EP 0125118. for example, U.S. Pat. No. 4,632,901, discloses a flow-through type immunoassay device comprising antibody (specific to a target antigen analyte) bound to a porous membrane or filter to which is added a liquid sample.

Detecting and determining the level of the p53 mRNA transcript may be performed e.g. by RT-PCR, oligonucleotide microarray or Northern blotting. Thus, the means may comprise PCR primers or probes specific to mutant p53, and optionally control primers or probes and/or one or more enzymes, buffers or other reagents used in these methods.

A brief description of non-limiting examples for methods detecting and quantifying RNA is provided hereinbelow.

Reverse transcriptase-polymerase chain reaction (RT-PCR) uses PCR amplification of relatively rare RNA molecules. First, RNA molecules are purified from cells and converted into complementary DNA (cDNA) using a reverse transcriptase enzyme (such as an MMLV-RT) and primers such as oligo-dT, random hexamers, or gene-specific primers. Then by applying gene-specific primers and Taq DNA polymerase, a PCR amplification reaction is carried out in a PCR machine. Those of ordinary skill in the art are capable of selecting the length and sequence of the gene-specific primers and the PCR conditions (i.e., annealing temperatures, number of cycles, and the like) that are suitable for detecting specific RNA molecules. It will be appreciated that a semi-quantitative RT-PCR reaction can be employed, by adjusting the number of PCR cycles and comparing the amplification product to known controls.

Real time RT-PCR follows the general principle of RT-PCR, with the difference being that the amplification product is measured in “real time” during the PCR reaction rather than at the end of the process. This enables the researcher to observe the amplification before any reagent becomes rate limiting for amplification.

Two common methods for detection of products in real-time PCR are: (1) non-specific fluorescent dyes that intercalate with any double-stranded DNA, and (2) sequence-specific DNA probes consisting of oligonucleotides that are labeled with a fluorescent reporter which permits detection only after hybridization of the probe with its complementary DNA target. Several fluorescence methodologies are available to measure amplification product in real-time PCR. Taqman (Applied BioSystems, Foster City, Calif.) uses fluorescence resonance energy transfer (FRET) to inhibit signal from a probe until the probe is degraded by the sequence specific binding and Taq 3′ exonuclease activity. Molecular Beacons (Stratagene, La Jolla, Calif.) also use FRET technology, whereby the fluorescence is measured when a hairpin structure is relaxed by the specific probe binding to the amplified DNA. The third commonly used chemistry is Sybr Green, a DNA-binding dye (Molecular Probes, Eugene, Oreg.). The more amplified product that is produced, the higher the signal. Other detection chemistries can also been used, such as ethidium bromide or other DNA-binding dyes and many modifications of the fluorescent dye/quencher dye Taqman chemistry, for example scorpions.

In the oligonucleotide microarray method, oligonucleotide probes capable of specifically hybridizing with the polynucleotides of interest, e.g. the p53 mRNA, are attached to a solid surface (e.g., a glass wafer). Each oligonucleotide probe is of approximately 20-25 nucleic acids in length. To detect the expression pattern of the polynucleotides of the present invention in a specific sample, RNA is extracted from the cell sample using methods known in the art (using, e.g., a TRIZOL® solution, Gibco-BRL™, USA). Hybridization can take place using either labeled oligonucleotide probes (e.g., 5′-biotinylated probes) or labeled fragments of complementary DNA (cDNA) or RNA (cRNA). Briefly, double-stranded cDNA is prepared from the RNA using reverse transcriptase (RT) (e.g., Superscript™ II RT), DNA ligase, and DNA polymerase I, all according to the manufacturer's instructions (Invitrogen Life Technologies, Frederick, Md., USA). To prepare labeled cRNA, the double-stranded cDNA is subjected to an in vitro transcription reaction in the presence of biotinylated nucleotides using, e.g., the BioArray™ HighYield™ RNA Transcript Labeling Kit (Enzo Diagnostics, Inc., Farmingdale, N.Y., USA). For efficient hybridization the labeled cRNA can be fragmented e.g. by incubating the RNA in 40 mM Tris Acetate (pH 8.1), 100 mM potassium acetate, and 30 mM magnesium acetate, for 35 minutes at 94° C. Following hybridization, the microarray is washed and the hybridization signal is scanned using a confocal laser fluorescence scanner, which measures fluorescence intensity emitted by the labeled cRNA bound to the probe arrays.

For example, commercially available p53-specific microarray is marketed by Affymetrix (GeneChip p53 assay). The GeneChip p53 assay uses more than 50,000 unique DNA probes synthesized on a glass “chip” to analyze p53 (exons 2-11) which includes the more than 400 distinct mutations that have been reported in the literature.

Northern blot analysis involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radioisotopes or enzyme-linked nucleotides. Detection may be performed by autoradiography, colorimetric reaction, or chemiluminescence. This method allows for both quantitation of an amount of a particular RNA molecule and determination of its identity by a relative position on the membrane which is indicative of a migration distance in the gel during electrophoresis.

Primer and probe sequences to be used in the above and other assays may be readily prepared by the skilled artisan based on p53 sequences known in the art. In a particular embodiment the p53 is human p53. Non-limitative examples of such sequences are exemplified below (see e.g. Table 1). Primers can be selected manually by the skilled artisan, or with the assistance of a computer implemented algorithm that optimizes primer selection based on desired parameters, such as annealing temperature, length, GC content, etc. Numerous computer implemented algorithms or programs for use via the internet or on a personal computer are available for these purposes, for example CODEHOP, DoPrimer, Primer3, Primer Selection, Web Primer, PCR Designer and others.

In other embodiments, the kits may further contain means for collecting a cell-containing sample from the subject, and/or means for purifying cells, nucleic acids or protein fractions from the sample. For example, isolation of proteins or RNA from the samples (and optional mRNA purification) may be performed by conventional methods known in the art (see e.g. Sambrook, J. and Russell, D. W. (2001), Molecular Cloning: A Laboratory Manual). Commercial kits for RNA extraction are also available, e.g. EZ-RNA (Biological Industry, Kibbutz Beit Haemek) and TRIZOL® (Gibco-BRL™, USA).

According to further embodiments, the kit may further comprise positive control samples (e.g. from cells expressing mutant p53) and negative control samples (e.g. from cells expressing native p53 or not expressing p53), or reference control values (corresponding to mean representative values in positive or negative control samples).

Polypeptide Synthesis

The polypeptides of the invention may be isolated or synthesized using any recombinant or synthetic method known in the art, including, but not limited to, solid phase (e.g. Boc or f-Moc chemistry) and solution phase synthesis methods. In alternate embodiments, polypeptides and peptides may be produced by recombinant technology. Recombinant methods for designing, expressing and purifying proteins and peptides are known in the art (see, e.g. Sambrook et al., 1989, 1992, 2001, Molecular Cloning: A Laboratory Manual, Cold Springs Harbor Laboratory, New York). Nucleic acid molecules may include DNA, RNA, or derivatives of either DNA or RNA. An isolated nucleic acid sequence encoding a polypeptide or peptide can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis. Nucleic acid sequences include natural nucleic acid sequences and homologs thereof, including, but not limited to, natural allelic variants and modified nucleic acid sequences in which nucleotides have been inserted, deleted, substituted, and/or inverted in such a manner that such modifications do not substantially interfere with the nucleic acid molecule's ability to encode a functional peptide. A polynucleotide or oligonucleotide sequence can be deduced from the genetic code of a protein, however, the degeneracy of the code must be taken into account, as well as the allowance of exceptions to classical base pairing in the third position of the codon, as given by the so-called “Wobble rules”. Polynucleotides that include more or less nucleotides can result in the same or equivalent proteins. Using recombinant production methods, selected host cells, e.g. of a microorganism such as E. coli or yeast, are transformed with a hybrid viral or plasmid DNA vector including a specific DNA sequence coding for the polypeptide or polypeptide analog and the polypeptide is synthesized in the host upon transcription and translation of the DNA sequence.

In various embodiments, the sequences may be derived directly from the corresponding sequence of the polypeptide (e.g. cytokine) or may contain certain derivatizations and substitutions. Thus in some embodiments the use of salts and functional derivatives of these sequences are contemplated, as long as they retain the respective biologic functions, as detailed herein. Accordingly the present invention encompasses peptide homologs containing non-natural amino acid derivatives or non-protein side chains. The peptide homologs of the invention may be used having a terminal carboxy acid, as a carboxy amide, as a reduced terminal alcohol or as any pharmaceutically acceptable salt, e.g., as metal salt, including sodium, potassium, lithium or calcium salt, or as a salt with an organic base, or as a salt with a mineral acid, including sulfuric acid, hydrochloric acid or phosphoric acid, or with an organic acid e.g., acetic acid or maleic acid. Generally, any pharmaceutically acceptable salt of the polypeptide of the invention may be used, as long as the biological activities of the polypeptide are maintained.

The amino acid residues described herein are preferred to be in the “L” isomeric form. However, residues in the “D” isomeric form can be substituted for any L-amino acid residue, as long as the peptide substantially retains the desired functional property.

Chemical derivatives may have one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhidstidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine may be substituted for lysine.

In addition, a derivative can differ from the natural sequence of the polypeptides or peptides of the invention by chemical modifications including, but are not limited to, terminal-NII₂ acylation, acetylation, or thioglycolic acid amidation, and by terminal-carboxlyamidation, e.g., with ammonia, methylamine, and the like.

According to some embodiments, the use of polypeptide homologs (or analogs) that differ from the native sequence by one or more conservative amino acid substitutions (having an equivalent charge and degree of hydrophobicity, e.g. Arg to Lys and Asp to Glu), thus retaining the structural and functional properties of said native sequence, is contemplated.

A “conservative amino acid substitution” is one in which the amino acid residue is replaced with an amino acid residue having a similar side chain Families of amino acid residues having similar side chains have been defined in the art. These families include amino acids with basic side chains (e. g., lysine, arginine, histidine), acidic side chains (e. g., aspartic acid, glutamic acid), uncharged polar side chains (e.g., glycine, asparagine, glutamin, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Pharmaceutical Compositions

According to various embodiments, the type I IFN used in the methods of the invention is formulated in the form of a pharmaceutical composition, which may further comprise one or more pharmaceutically acceptable carriers, excipients and/or diluents.

In another embodiment there is provided a pharmaceutical composition comprising a type I IFN in unit dosage form, at a dose that does not substantially induces apoptotic cell death when administered to a subject in need thereof.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes. A pharmaceutical composition of the invention is formulated to be compatible with its intended route of administration. Methods to accomplish the administration are known to those of ordinary skill in the art. Examples of suitable excipients and modes for formulating the compositions are described in the latest edition of “Remington's Pharmaceutical Sciences” by E. W. Martin.

For example, saline solutions and aqueous dextrose and glycerol solutions can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Solutions or suspensions used for intravenous administration typically include a carrier such as physiological saline, bacteriostatic water, Cremophor EL™ (BASF, Parsippany, N.J.), ethanol, or polyol. In all cases, the composition must be sterile and fluid for easy syringability.

Proper fluidity can often be obtained using lecithin or surfactants. The composition must also be stable under the conditions of manufacture and storage. Prevention of microorganisms can be achieved with antibacterial and antifungal agents, e.g., parabens, chlorobutanol, phenol, ascorbic acid, thimerosal, etc. In many cases, isotonic agents (sugar), polyalcohols (mannitol and sorbitol), or sodium chloride may be included in the composition. Prolonged absorption of the composition can be accomplished by adding an agent which delays absorption, e.g., aluminum monostearate and gelatin. Where necessary, the composition may also include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Oral compositions include an inert diluent or edible carrier. The composition can be enclosed in gelatin or compressed into tablets. For the purpose of oral administration, the active agent can be incorporated with excipients and placed in tablets, troches, or capsules.

Pharmaceutically compatible binding agents or adjuvant materials can be included in the composition. The tablets, troches, and capsules, may optionally contain a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; or a sweetening agent or a flavoring agent.

Nucleic Acids

In some embodiments, the invention relates to methods in which, if a subject is identified as being amenable for treatment with a type I IFN as described herein, the method further comprises expressing in cells of said subject a type I IFN.

According to some embodiments, expressing in cells of said subject a type I IFN is performed by administering to cells of a subject a nucleic acid construct comprising a nucleic acid sequence encoding a type I IFN that is operably linked to a transcription regulating sequence.

In another aspect the invention provides a nucleic acid construct for use in the treatment or prevention of a neoplastic disorder,

wherein:
the construct comprises a nucleic acid sequence encoding a type I IFN, which nucleic acid sequence is operably linked to a transcription-regulating sequence, and
the subject is identified as expressing mutant p53 that is characterized by one or more mutations residing within the DNA binding domain.

The phrase “operably linked” refers to a nucleic acid sequence linked a to a transcription control sequence in a manner such that the molecule is able to be expressed when transfected (i.e., transformed, transduced, infected, or transfected) into a host cell. Transcription control sequences are sequences, which control the initiation, elongation, and termination of transcription. Particularly important transcription control sequences are those which control transcription initiation, such as promoter, enhancer, operator and repressor sequences.

It will be appreciated that the IFN polypeptides of the present invention may be expressed directly in the subject (i.e. in vivo gene therapy) or may be expressed ex vivo in a cell system (autologous or non-autologous) and then administered to the subject. Thus, in another embodiment, the step of expressing in cells of said subject a type I IFN is performed in vivo, and in another embodiment, the step of expressing in cells of said subject a type I IFN is performed ex vivo.

To express such an agent (i.e., to produce an IFN polypeptide) in mammalian cells, a nucleic acid sequence encoding the agents is preferably ligated into a nucleic acid construct suitable for mammalian cell expression. Such a nucleic acid construct includes a promoter sequence for directing transcription of the polynucleotide sequence in the cell in a constitutive or inducible manner.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the tetracycline-inducible promoter.

The nucleic acid construct (also referred to herein as an “expression vector”) of the present invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of the present invention is active in the specific cell population transformed. Examples of cell type-specific and/or tissue-specific promoters include promoters that are lymphoid specific promoters, in particular promoters of T-cell receptors and immunoglobulins.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine CMV, the long term repeat from various retroviruses such as murine leukemia virus, murine or RSV.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase RNA stability. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples for mammalian expression vectors include, but are not limited to, pcDNA3, pcDNA3.1(+/−), pGL3, pZeoSV2(+/−), and expression vectors containing regulatory elements from eukaryotic viruses such as pMSG, pAV009/A⁺, pMTO10/A⁺, pMAMneo-5, and baculovirus pDSVE.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in and include, for example, stable or transient transfection, lipofection, electroporation and infection with recombinant viral vectors. In addition, see U.S. Pat. Nos. 5,464,764 and 5,487,992 for positive-negative selection methods. Exemplary in vivo nucleic acid transfer techniques include transfection with viral or non-viral constructs, such as adenovirus, lentivirus, Herpes simplex I virus, or adeno-associated virus (AAV) and lipid-based systems.

Conveniently, a nucleic acid construct comprising a nucleic acid sequence encoding a type I IFN that is operably linked to a transcription regulating sequence, may be administered to cells of the subject in the form of a nucleic acid composition, further comprising a pharmaceutically acceptable carrier. With respect to nucleic acid compositions, a “carrier” refers to any substance suitable as a vehicle for delivering a nucleic acid sequence of the present invention to a suitable in vivo site. As such, carriers can act as a pharmaceutically acceptable excipient of a therapeutic composition containing a nucleic acid molecule of the present invention. Preferred carriers are capable of maintaining a nucleic acid molecule of the present invention in a form that, upon arrival of the nucleic acid molecule to a cell, the nucleic acid molecule is capable of entering the cell and being expressed by the cell. Carriers nucleic acid compositions of the present invention may include: (1) excipients or formularies that transport, but do not specifically target a nucleic acid molecule to a cell (referred to herein as non-targeting carriers); and (2) excipients or formularies that deliver a nucleic acid molecule to a specific site in an animal or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- and o-cresol, formalin and benzol alcohol. Other carriers can include metal particles (e.g., gold particles) for use with, for example, a biolistic gun through the skin. Therapeutic compositions of the present invention can be sterilized by conventional methods.

For example, without limitation, a single dose of a nucleic acid construct encoding a type I IFN according to certain embodiments of the invention in a non-targeting carrier for in vivo administration, may be from 12.5 μg to 20 mg of total recombinant molecules per kg body weight. It will be obvious to one of skill in the art that the number of doses administered is dependent upon the extent of the disease and the response of an individual patient to the treatment. Thus, it is within the scope of the present invention that a suitable number of doses includes any number required to cause regression of a disease.

### Additional Exemplary Embodiments

Certain additional exemplary embodiments of the invention are described below.

1. A type I IFN for use in the treatment of a subject identified as expressing mutant p53.

2. A type I IFN for use in the treatment of a subject identified as expressing mutant p53 that is characterized by one or more mutations residing within the DNA binding domain. 3. A type I IFN for use in the treatment or prevention of a neoplastic disorder in a subject identified as expressing mutant p53 that is characterized by one or more mutations residing within the DNA binding domain. 4. A type I IFN for use in the treatment of a subject afflicted with a disorder associated with a p53 mutation, wherein the subject is identified as being amenable for treatment with the type I IFN if said subject is determined to express mutant p53 that is characterized by one or more mutations residing within the DNA binding domain.

5. The type I IFN for use according to any one of clauses 1-4, wherein said IFN is an IFN-β or an IFN-α. 6. The type I IFN for use according to any one of clauses 1-4, wherein said IFN is IFN-β. 7. The type I IFN for use according to any one of clauses 1-4, wherein said IFN is IFN-β, preferably IFN-β1. 8. The type I IFN for use according to any one of clauses 1-4 wherein said IFN is IFN-β1 preferably human IFN-β1. 9. The type I IFN for use according to any one of clauses 1-4, wherein said IFN is a mammalian IFN-β or IFN-α, preferably human IFN-β or IFN-α, more preferably human IFN-β. 10. The type I IFN for use according to any one of clauses 1-4, wherein said IFN is IFN-α, preferably IFN-α2, more preferably human IFN-α2. 11. The type I IFN for use according to clause 10, wherein the IFN-α2 differs from native IFN-α2 by at least two substitutions, selected from the group consisting of: (a) substitution of the histidine at position 57 (H57) to a residue selected from the group consisting of tyrosine and methionine; (b) substitution of the glutamate at position 58 (E58) to a residue selected from the group consisting of asparagine, aspartate, leucine, and alanine; (c) substitution of the glutamine at position 61 (Q61) to a residue selected from the group consisting of serine, leucine, and aspartate; and (d) substitution of the 5-10 C-terminal residues of said IFN-α2 to a sequence selected from the group consisting of KRLKSKE (SEQ ID NO: 41) and KRLKSK (SEQ ID NO: 42). 12. The type I IFN for use according to clause 10, wherein the IFNα2 polypeptide comprises substitutions H57Y, E58N, and Q61S in native human IFNα2, and optionally further comprises a substitution of the C terminal ESLRSKE to KRLKSKE (SEQ ID NO: 41).

13. The type I IFN for use according to any one of the preceding clauses wherein determining whether the subject expresses mutant p53 is performed by determining the presence of mutant p53 protein or of mutant p53 mRNA transcript. 14. The type I IFN for use according to any one of the preceding clauses, wherein determining whether the subject expresses mutant p53 is performed by determining the presence of mutant p53 mRNA, wherein if said subject expresses mutant p53 mRNA, said subject is determined to be amenable for treatment with the type I IFN. 15. The type I IFN for use according to any one of the preceding clauses, wherein determining whether the subject expresses mutant p53 further comprises determining mutant p53 mRNA levels, wherein if said subject expresses mutant p53 mRNA, and the level of the mutant p53 mRNA is reduced upon type I IFN treatment, said subject is determined to be amenable for treatment with said type I IFN.

16. The type I IFN for use according to any one of the preceding clauses, wherein the subject is afflicted with or is predisposed to developing a neoplastic disorder. 17. The type I IFN for use according to clause 1 wherein said disorder is Li-Fraumeni syndrome or another disorder associated with a p53 germline mutation. 18. The type I IFN for use according to any one of the preceding clauses wherein said disorder is associated with a p53 germline mutation.

19. The type I IFN for use according to clause 18 wherein said disorder is Li-Fraumeni syndrome. 20. The type I IFN for use according to clause 16 wherein the disorder is cancer or cancer metastasis. 21. The type I IFN for use according to clause 16 wherein the disorder is cancer. 22. The type I IFN for use according to clause 16 wherein the disorder is cancer metastasis. 23. The type I IFN for use according to clause 1 or 16 wherein said subject is a cancer patient in a state of remission.24. The type I IFN for use according to clause 1 or 16 wherein said subject is predisposed to developing the disorder. 25. The type I IFN for use according to any one of the preceding clauses wherein said type I IFN is formulated for in vivo administration. 26. The type I IFN for use according to any one of the preceding clauses wherein said type I IFN is formulated for ex vivo administration. 27. The type I IFN for use according clause 1 or 16 for sensitizing a chemotherapy-resistant tumor to chemotherapy. 25. The type I IFN for use according to clause 1 or 16 for reducing cellular levels of mutant p53, thereby treating or preventing cancer or metastasis in said subject. 26. The type I IFN for use according to clause 1 or 16 for reducing cellular levels of mutant p53, thereby treating said subject. 30. The type I IFN for use according to clause 1 or 16 for reducing cellular levels of mutant p53, thereby treating Li-Fraumeni syndrome in said subject. 31. The type I IFN for use according to clause 1 or 16 for reducing cellular levels of mutant p53, thereby sensitizing a chemotherapy-resistant tumor to chemotherapy.

32. The type I IFN for use according to any one of the preceding clauses formulated as a pharmaceutical composition comprising said type I IFN in unit dosage form, at a dose that does not substantially induce apoptotic cell death when administered to said subject. 33. The type I IFN for use according to clause 32 wherein said IFN is IFN-β1. 34. The type I IFN for use according to any one of the preceding clauses for use as a sole active ingredient. 35. The type I IFN for use according to any one of the preceding clauses, wherein said mutant p53 is characterized by one or more mutations that down-regulate the level of said p53 mRNA upon exposure to type I IFN.

36. A nucleic acid construct for use in the treatment of a subject afflicted with a disorder associated with a p53 mutation, wherein: the construct comprises a nucleic acid sequence encoding a type I IFN that is operably linked to a transcription regulating sequence, and the subject is identified as being amenable for treatment with the type I IFN if said subject is determined to express mutant p53 that is characterized by one or more mutations residing within the DNA binding domain.

37. A nucleic acid construct for use in the treatment or prevention of a neoplastic disorder, wherein: the construct comprises a nucleic acid sequence encoding a type I IFN, which nucleic acid sequence is operably linked to a transcription-regulating sequence, and the subject is identified as expressing mutant p53 that is characterized by one or more mutations residing within the DNA binding domain.

38. The nucleic acid construct for use according to clause 36 or 37, wherein said construct is formulated for in vivo administration. 39. The nucleic acid construct for use according to clause 36 or 37, wherein said construct is formulated for ex vivo administration.

40. The nucleic acid construct for use according to clause 36 or 37, wherein said IFN is IFN-β or an IFN-α, preferably IFN-β, more preferably IFN-β1. 41. The nucleic acid construct for use according to clause 36 or 37, wherein the subject is afflicted with or is predisposed to developing a neoplastic disorder, preferably wherein said disorder is Li-Fraumeni syndrome, cancer or cancer metastasis.

42. A method comprising the steps of: a) determining whether a subject expresses mutant p53; and b) if the subject expresses mutant p53, identifying said subject as amenable for treatment with a type I IFN.

43. A method comprising the steps of: a) determining whether a subject expresses mutant p53 characterized by one or more mutations residing within the DNA binding domain; and b) if the subject expresses the mutant p53, identifying said subject as amenable for treatment with a type I IFN.

44. A method for determining whether a subject in need thereof is amenable for treatment with a type I IFN, comprising the steps of: a) determining whether the subject expresses mutant p53 characterized by one or more mutations residing within the DNA binding domain; and b) if the subject expresses the mutant p53, identifying said subject as amenable for treatment with the type I IFN.

45. A method for determining whether a subject in need thereof is amenable for treatment with a type I IFN, comprising the steps of: a) determining whether a subject expresses oncogenic mutant p53 characterized by one or more mutations that down-regulate the level of the p53 mRNA upon exposure to type I IFN; and b) if the subject expresses the mutant p53, identifying said subject as amenable for treatment with a type I IFN.

46. A method for determining whether a subject is amenable for treatment with a type I interferon (IFN), comprising the steps of: a) determining whether the subject expresses mutant p53 characterized by one or more mutations residing within the DNA binding domain; and b) if the subject expresses the mutant p53, identifying said subject as amenable for treatment with the type I IFN; wherein said treatment with said type I IFN is used for reducing cellular levels of mutant p53.

47. The method of any one of clauses 42-46, wherein the type I IFN is an IFN-β or an IFN-α. 48. The method of clause 47, wherein said IFN is IFN-β. 49. The method of clause 48, wherein said IFN is IFN-β1. 50. The method of clause 48, wherein said IFN is mammalian IFN-β, preferably human IFN-β, more preferably human IFN-β1. 51. The method of clause 47, wherein the type I IFN is an IFN-α, preferably IFN-α2.

52. The method of any one of clauses 42-51, wherein determining whether the subject expresses mutant p53 is performed by determining the presence of mutant p53 protein or of mutant p53 mRNA transcript. 53. The method of clause 52, wherein determining whether a subject expresses mutant p53 is performed by determining the presence of mutant p53 mRNA, wherein if said subject expresses mutant p53 mRNA, said subject is determined to be amenable for treatment with the type I IFN. 54. The method of any one of clauses 42-51, wherein determining whether the subject expresses mutant p53 further comprises determining mutant p53 mRNA levels, wherein if said subject expresses mutant p53 mRNA, and the level of the mutant p53 mRNA is reduced upon type I IFN treatment, said subject is determined to be amenable for treatment with the type I IFN.

55. The method of any one of clauses 42-54, wherein the subject is afflicted with or is predisposed to developing a neoplastic disorder. 56. The method of clause 55, wherein said disorder is Li-Fraumeni syndrome or another disorder associated with a p53 germline mutation.

57. The method of clause 55, wherein the disorder is cancer, cancer metastasis, or another disorder characterized by mutation in the p53 gene. 58. The method of clause 55, wherein said subject is a cancer patient in a state of remission. 59. The method of clause 55, wherein said subject is predisposed to developing the disorder.

60. An ex-vivo method according to any one of clauses 42-59. 61. A non-invasive method according to any one of clauses 42-59, wherein step a) is performed ex vivo.

62. The method of any one of clauses 42-61, wherein if said subject is identified as being amenable for treatment with the type I IFN, the method further comprises the step of: c) administering an effective amount of said type I IFN to said subject identified as being amenable for treatment with said type I IFN.

63. The method of any one of clauses 42-61, wherein if said subject is identified as being amenable for treatment with the type I IFN, the method further comprises the step of: c) contacting cells of said subject with said type I IFN. 64. The method of clause 63, wherein step c) is performed ex vivo.

65. The method of any one of clauses 42-61, wherein if said subject is identified as being amenable for treatment with the type I IFN, the method further comprises the step of: c) expressing in cells of said subject said type I IFN. 66. The method of clause 65, wherein step c) is performed ex vivo.

66. The method of any one of clauses 62-65, wherein the type I IFN is an IFN-β or an IFN-α. 67. The method of clause 66, wherein said IFN is IFN-β, preferably IFN-β1.

68. The method of any one of clauses 62-65, wherein said IFN is mammalian IFN-β, preferably human IFN-β, more preferably human IFN-β1. 69. The method of any one of clauses 62-65, wherein the type I IFN is an IFN-α, preferably IFN-α2.

70. A kit for identifying a subject as being amenable for treatment with a type I IFN, comprising: a) means for determining whether the subject expresses mutant p53; and b) instructions for identifying said subject as amenable for treatment with a type I IFN if said subject expresses mutant p53.

71. The kit of clause 70, wherein said IFN is IFN-β. 72. The kit of clause 71, wherein said IFN is IFN-β1. 73. The kit of clause 70, wherein said IFN is IFN-β, preferably mammalian IFN-β, more preferably human IFN-β. 74. The kit of clause 70, wherein said IFN is mammalian IFN-β, preferably human IFN-β, more preferably human IFN-β1. 75. The kit of clause 70, wherein the type I IFN is an IFN-α, preferably IFN-α2.

76. The kit according to any one of clauses 70-75, wherein the mutant p53 is characterized by one or more mutations residing within the DNA binding domain. 77. The kit according to any one of clauses 70-75, wherein the mutant p53 is characterized by one or more mutations that down-regulate the level of the p53 mRNA upon exposure to type I IFN.

78. The kit according to any one of the preceding clauses, wherein the subject is afflicted with or is predisposed to developing a neoplastic disorder. 79. The kit according to clause 78, wherein the disorder is cancer and/or metastases and/or is characterized by mutation in the p53 gene. 80. The kit according to clause 78, wherein said disorder is Li-Fraumeni syndrome.

81. The kit according to any one of the preceding clauses, comprising instructions for administering said type I IFN to said subject at therapeutically effective dose that ensures efficacy while minimizing side effects associated with the induction of apoptosis.

82. A pharmaceutical pack, comprising: a) a therapeutically effective dose of a type I IFN; and b) instructions for administering said type I IFN to a subject identified as expressing mutant p53.

83. The pharmaceutical pack of clause 82, wherein the mutant p53 is characterized by one or more mutations residing within the DNA binding domain. 84. The pharmaceutical pack of clause 82, wherein the mutant p53 is characterized by one or more mutations that down-regulate the level of the p53 mRNA upon exposure to type I IFN.

83. The pharmaceutical pack of any one of the preceding clause, wherein the subject is afflicted with or is predisposed to developing a neoplastic disorder. 84. The pharmaceutical pack of clause 83, wherein the disorder is cancer and/or metastases and/or is characterized by mutation in the p53 gene. 85. The pharmaceutical pack of clause 83, wherein the disorder is cancer. 86. The pharmaceutical pack of clause 83, wherein the disorder is cancer metastases. 87. The pharmaceutical pack of clause 83, wherein the disorder is characterized by mutation in the p53 gene. 88. The pharmaceutical pack of clause 87 wherein said disorder is Li-Fraumeni syndrome.

89. The pharmaceutical pack of any one of the preceding clauses, wherein said therapeutically effective dose ensures efficacy while minimizing side effects associated with the induction of apoptosis. 90. The pharmaceutical pack of any one of the preceding clauses, wherein said IFN is IFN-β. 91. The pharmaceutical pack of any one of the preceding clauses, wherein said IFN is IFN-β1. 92. The pharmaceutical pack of any one of clauses 82-91, wherein the type I IFN is an IFN-α, preferably IFN-α2.

The following examples are presented in order to more fully illustrate some embodiments of the invention. They should, in no way be construed, however, as limiting the broad scope of the invention.

### EXAMPLES

Materials and Methods

Cell lines: HK3-T lung CAFs cells were cultured in a humidified incubator at 37° C. and 5% CO₂. CAFs and HepG2 (hepatocellular carcinoma) were grown in MEM, H1299 (non-small cell lung carcinoma) and SKBR-3 (breast adenocarcinoma) in RPMI, A549 (lung adenocarcinoma) in DMEM and HCT116 (colorectal carcinoma) in McCoy's Media supplemented with 10% FCS and Pen/Strep solution (Biological industries, Beit-Haemek, Israel).

Western blot Analysis: Total cell extracts were fractionated by gel electrophoresis; proteins were transferred to nitrocellulose membranes, and immunoblotted using the designated antibodies: anti-GAPDH mab374, (Chemicon, Billerica, Mass.) and anti-p53 DO1. The protein-antibody complexes were detected by horseradish peroxidase-conjugated secondary antibodies followed by the enhanced SuperSignal west pico chemiluminescent substrate (Thermo scientific, IL, USA).

Isolation of Total RNA and Quantitative Real-Time PCR (QRT-PCR): Total RNA was isolated using the NucleoSpin RNA II kit (Macherey-Nagel), according to the manufacturer's protocol. A 2ug aliquot was reverse transcribed using MMLV-RT (Bio-RT) and random hexamer primers. QRT-PCR was performed on an ABI 7300 instrument (Applied Biosystems) using Platinum SYBR Green and qPCR SuperMix (Invitrogen). Primers sequences are listed in Table 1 below. Data analysis was performed according to the ΔΔCt method using HPRT as the endogenous control. The results are presented as a mean±S.D. of two or three duplicate runs from a representative experiment.

TABLE 1
QRT-PCR primers

| | Forward primer | Reverse primer |
|---|---|---|
| Target | (SEQ ID NO.) | (SEQ ID NO.) |
| IFNα* | CCCTCTCTTTATCAAC | TTGTTTTCATGTTGGA |
| | AAACTTGC (10) | CCAGA (11) |
| IFNβ* | CGACACTGTTCGTGTT | GAAGCACAACAGGAGA |
| | GTCA (12) | GCAA (13) |
| MX1 | GTTTCCGAAGTGGACA | CCATTCAGTAATAGAG |
| | TCGCA (14) | GGTGGGA (15) |
| STAT1 | ATGTCTCAGTGGTACG | TGCTCCAGGAATTTTG |
| | AACTTCA (16) | AGTCAAG (17) |
| SOCS1* | CCCCTGGTTGTTGTAG | GTAGGAGGTGCGAGTT |
| | CAG (18) | CAGG (19) |
| p53 | CCCAAGCAATGGATGA | GGCATTCTGGGAGCTT |
| | TTTGA (20) | CATCT (21) |
| WIG1 | CGGCAGAGAATTCCAC | ATCTCTTCGCCAGCTC |
| | GTGAT (22) | CAACA (23) |
| GFP | GGGCACAAGCTGGAGT | CACCTTGATGCCGTTC |
| | ACAAC (24) | TTCTG (25) |
| dsRed | GCTCCTCCAAGAACGT | GGGTGCTTCACGTACA |
| | CATC (26) | CCTT (27) |
| SP1 | AAACGTACACACACAG | CTCCTCATGAAGCGC |
| | GTGAGAAGA (28) | TTAGGA (29) |
| p21 | GGCAGACCAGCATGAC | GCGGATTAGGGCTTC |
| | AGATT (30) | CTCT (31) |
| ELN | GCAGGAGTTAAGCCCA | TGTAGGGCAGTCCAT |
| | AGG (32) | AGCCA (33) |
| PDGFB | TTAAGAAGGCCACGGT | TCTCACACTTGCATG |
| | GACG (34) | CCAGG (35) |
| CXCL11 | GACGCTGTCTTTGCAT | GGATTTAGGCATCGT |
| | AGGC (36) | TGTCCTTT (37) |
| IFIT1 | AAGCTTTCAAATCCCT | GCCTTGGCCCGTTCA |
| | TCCGC (38) | TAATT (39) |

*Roche taqman primers

Image Stream FACS: Cells were collected, trypsinized and supplemented with 5 mM EDTA, washed and reconstituted in 70% ETOH-HBSS and incubated for 1 hr in −20° c. Cells were then blocked with 3%BSA-PBS and supplemented with the designated antibodies: anti-p53 DO-1, anti-STAT1 p91, C-24:sc-456 and anti-pSTAT1 Tyr 701:sc-7988 (Santa-cruz). Cells were then washed and supplemented with fluorescent antibodies (DAPI, cy3 and cy5). After washing, cells were centrifuged and reconstituted in 100 μl sorted and analyzed. As controls, each dye was measured alone and its penetration to other channels was deducted from all other channels. For nuclear localization, the similarity between the mean intensity of DAPI and the desired protein was calculated.

Interferons treatment: Recombinant human Interferon α (#300-02-AB), β (#300-02BC-100), and γ (#300-02-100), and their corresponding antibodies: α (500-P32A), β (500-P32B), and γ (500-P32), were purchased from Peprotech, Israel. IFNs concentrations used in this study were as follows: IFN-α-1000 units/ml, IFN-β-1 nM, and IFNγ 10 ng/ml.

SOCS1 knockdown: Cells (5×10⁴) were seeded in a 6 cm plate and were treated with siRNA against either SOCS1 or LacZ as a control according to the manufacturer protocol (Thermo scientific) for 48 hrs.

Statistical analysis: Unless stated otherwise, an unpaired one-tailed student t-test was performed. *denotes at least p<0.05.

cDNA Microarray: Total RNA was extracted using Tri-Reagent (MRC Inc.) according to manufacturer's protocol, and sent to the MicroArray unit (Weizmann institute of science, Rehovot, Israel). Agilent chips (IIuman 8×60K) were used as a platform for RNA loading. Each sample expression was compared to a common reference sample comprised of an equal amount of RNA from all samples. The limma package was used for microarray processing. Background was corrected using the function background Correct and normalization within and between arrays was performed using the functions normalize Within Arrays and normalize Between Arrays, respectively. Spots with the same probes were averaged. Analysis of variance (ANOVA) including contrasts was applied to the data set using Partek Genomic Suite 6.5 (Inc. St. Charles, Mo.). The microarray data have been submitted to the Gene Expression Omnibus (GEO) database and assigned the identifier accession GSE41477.

### Example 1

### Establishment of an In Vitro Model to Study the Tumor-Stroma Encounter in Lung Cancer

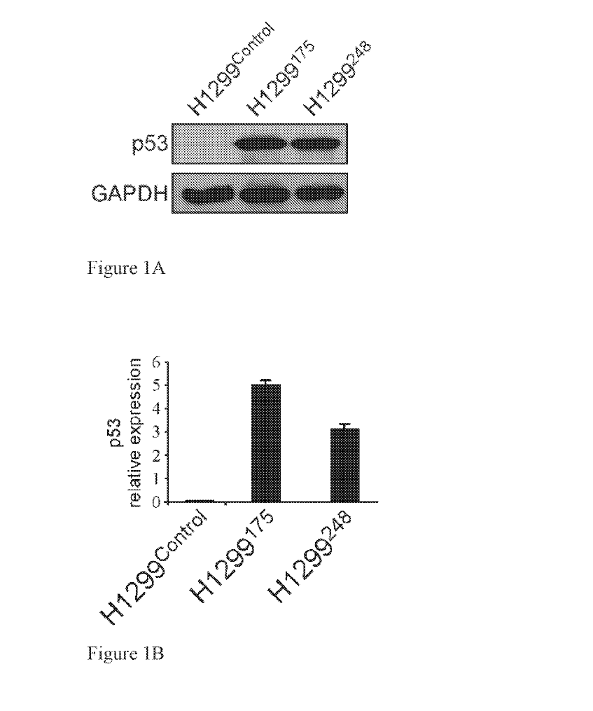

As stromal cells often reside in, or are recruited to the vicinity of the tumor, an in vitro co-cultivation model was established that recapitulates this encounter and permits an efficient separation and characterization of the two cell populations. Lung cancer cells (H1299) which are null for p53 expression were introduced with two p53 totspof mutations residing within the DNA binding domain, namely R175H and R248Q (H1299¹⁷⁵ and H1299²⁴⁸ respectively, FIGS. 1A and B). The cells were then labeled with a red fluorescent protein (dsRed), while lung CAFs (HK3-T) were labeled with a green fluorescent protein (GFP). The labeled populations were co-cultivated for 24 hours and separated by Fluorescence Associated Cell Sorting (FACS) based on their specific fluorescent marker. To minimize the possibility of cross contamination, the separated populations were sorted again, and indeed, the level of cross contamination was diminished. To further corroborate this observation, quantitative real time PCR (QRT-PCR) was performed with primers amplifying either GFP or dsRed. Following the double-sorting procedure, GFP and dsRed expression was several orders of magnitude higher in the corresponding labeled cells. Because the sorting procedure includes prolonged incubation on ice, and cells may be subjected to mechanical stress introduced by the FACS machinery, the expression levels of stress-related genes were measured prior and post the sorting procedure.

First, p21, a common stress response gene, was found to be expressed in a comparable manner in the sorted and unsorted samples. Several other genes that are known to be specifically elevated during mechanical stress in lung cells were found to be either equally expressed or down regulated following the sorting procedure.

Taken together, these results indicate that the novel experimental system described herein is capable of separating the two cell populations with a high degree of purity, without imposing measurable mechanical stress.

### Example 2

### CAFs Invoke the Interferon Beta Pathway in Response to the Presence of Cancer Cells

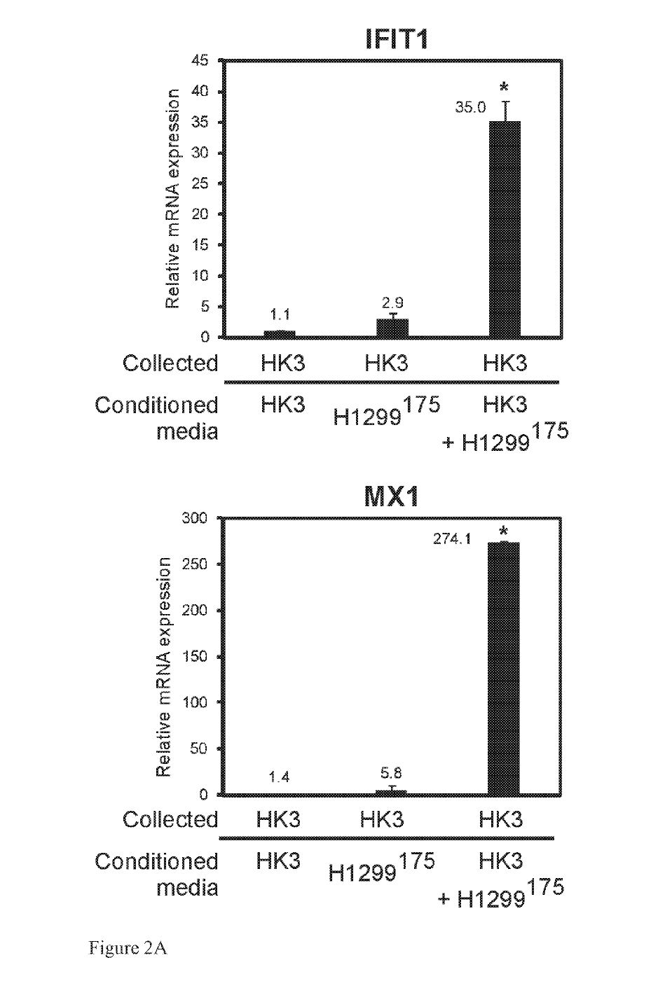

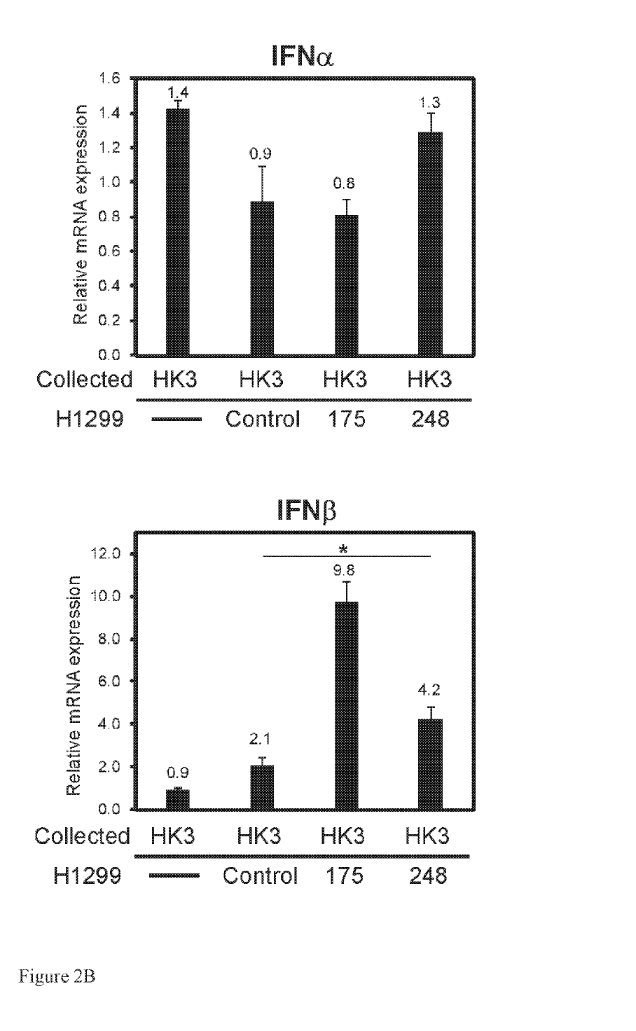

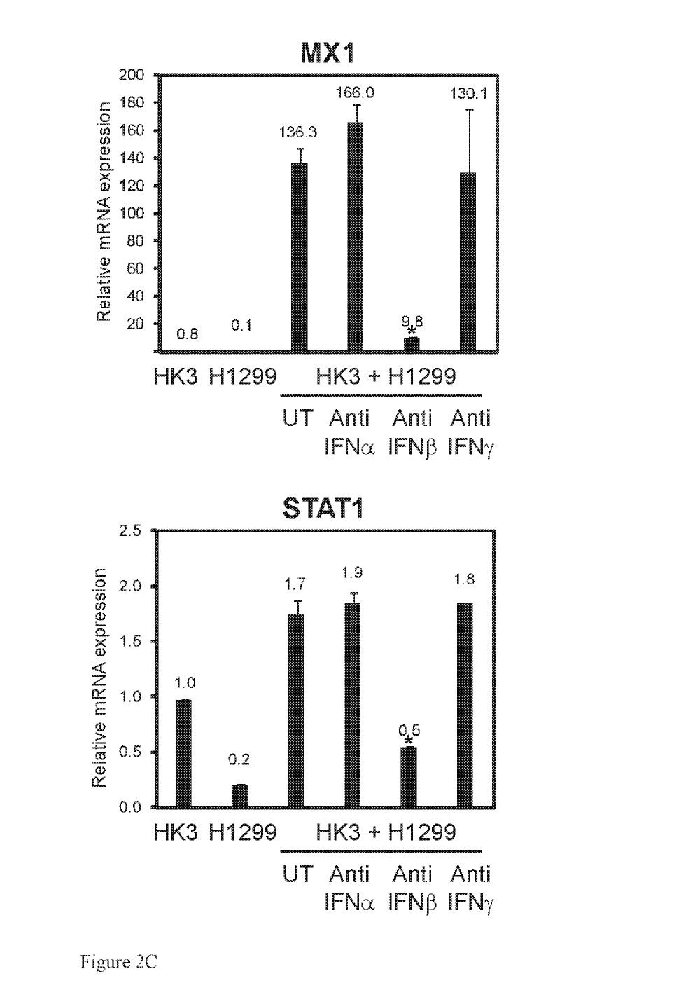

To gain insights into the gene expression profile of stromal cells following the encounter with mutant p53 expressing cancer cells, the differentially expressed genes (two fold change or more, 0.05 p-value or less) in HK3-T before and after co-cultivation with either p53 null, H1299¹⁷⁵ or H1299²⁴⁸ cancer cells via micro-array were analyzed. This comparison yielded a list of 875 differentially expressed genes that were clustered into 8 distinct groups by the CLICK algorithm using the Expander package (version 5.2). Of note, is the first cluster (‘HK3-T cluster’) composed of a group of 414 genes induced by the mere co-cultivation with carcinoma cells. This induction was further enhanced in the presence of mutant p53 expressing cells. The ‘HK3-T cluster’ was further characterized by the use of IPA algorithm (Ingenuity® Systems) which identifies enriched Gene Ontology annotations and canonical pathways within a given list of genes. The most significantly enriched term was the “interferon signaling pathway”, for which 14 genes out of 36 were elevated in HK3-T (p-value-8.4×10⁻¹⁶) in response to co-cultivation with carcinoma cells. In a study by Buess and colleagues, breast stromal cells and breast cancer cell lines were co-cultivated and subjected to micro-array analysis, and the most significant cluster was enriched with an IFN signature consisting of 31 genes. This ‘interferon cluster’ was compared with the ‘HK3-T cluster’ and yielded an overlap of 24 out of 31 genes. In addition, the ‘HK3-T cluster’ was compared with a database of ˜2000 known IFNs targets termed ‘interferome’ (Samarajiwa et al) and an enrichment of 37% (152 out of 414) was observed. It was then investigated whether physical interaction between the cells is a prerequisite for triggering the IFN pathway or whether carcinoma cells grown alone are able to secrete factors, which evoke such a response without the presence of CAFs. Conditioned media collected from carcinoma cells grown alone induced a slight elevation of IFN targets in CAFs (FIG. 2A). A more prominent effect however, was observed when conditioned media collected from a co-culture of CAFs and carcinoma cells was transferred to the CAFs.

To rule out the possibility of cell line specific effects, several other combinations of CAFs and lung carcinoma cells were compared. HK3 were able to induce the IFN pathway when co-cultivated with the carcinoma cell line A549, but not with H460. CAFs derived from another patient were able to evoke the IFN pathway as well, however, not when co-cultured with each other. These data suggest that the IFN pathway is up regulated in some but not all pairs of CAFs and lung carcinoma cells and not in the presence of normal cells. All IFNs share mutual targets, and more specifically type I IFNs (α and β) are almost inextricable with regards to their targets, and mainly differ by their affinity to the type I IFN receptors. To differentiate between the IFNs and reveal the identity of the predominant cytokine in the instant experimental model, the mRNA expression levels of IFNs α, β and γ were compared. IFN γ was not detected in CAFs, regardless of the presence of carcinoma cells. IFN α levels were comparable between the samples, whereas IFN β levels were elevated in CAFs when cultivated with carcinoma cells. In the presence of carcinoma cells expressing mutant p53, IFN-β levels were further induced in accordance with our microarray results (FIG. 2B). As IFNs are secreted cytokines, the IFN effect was antagonized by administering antibodies against IFN s α, β and γ. To that end, an IFN response was initiated by co-cultivating CAFs and carcinoma cells, leading to the elevation of IFN targets MX1 and STAT. This elevation was exclusively abolished by the addition of anti-IFN β antibody, and not in the presence of anti-IFN α or γ antibodies (FIG. 2C).

Combined, these data suggest that CAFs secrete IFN beta upon their encounter with carcinoma cells.

### Example 3

### Mutant p53-Bearing Cells Moderate CAFs-Mediated Interferon Response

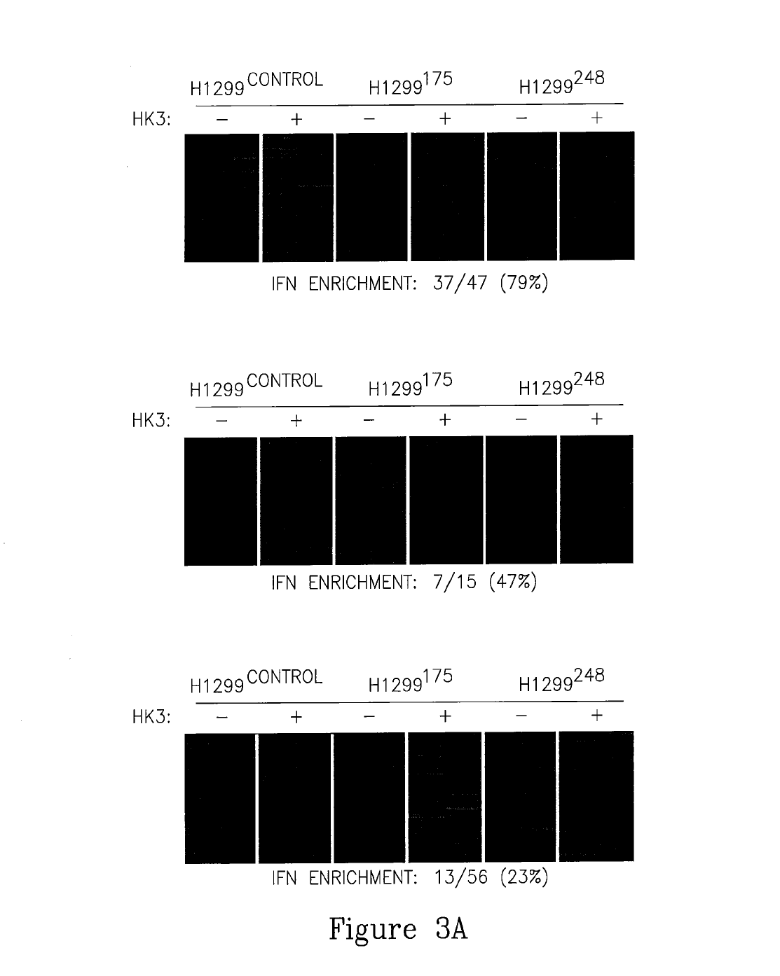

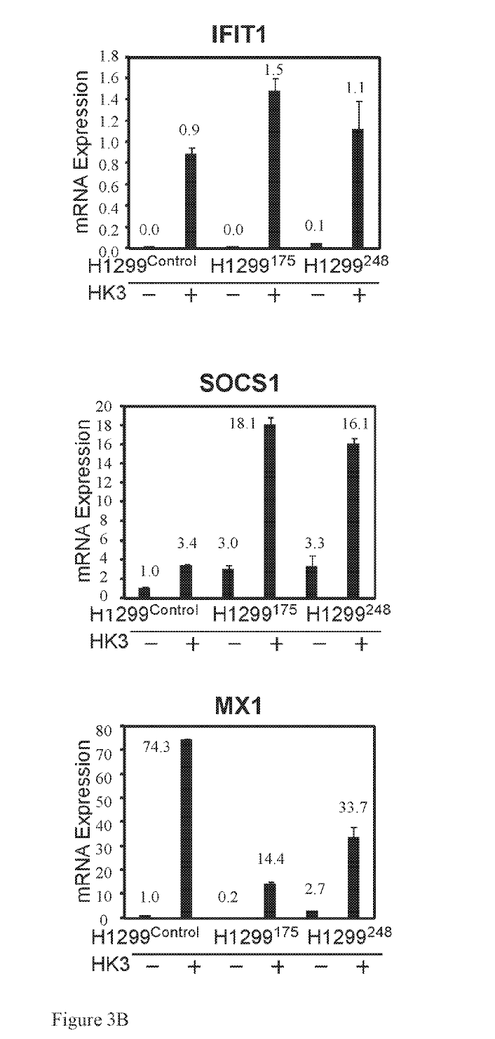

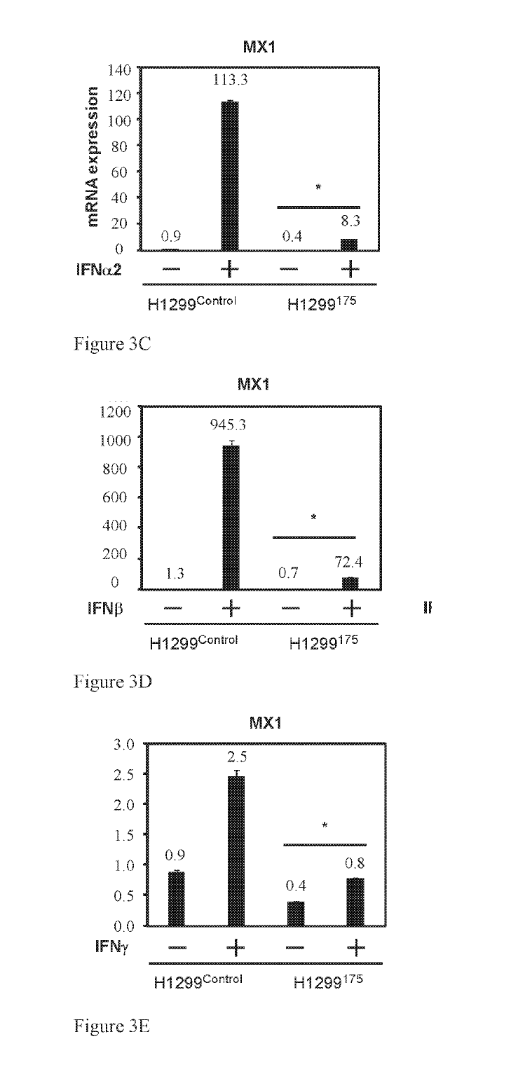

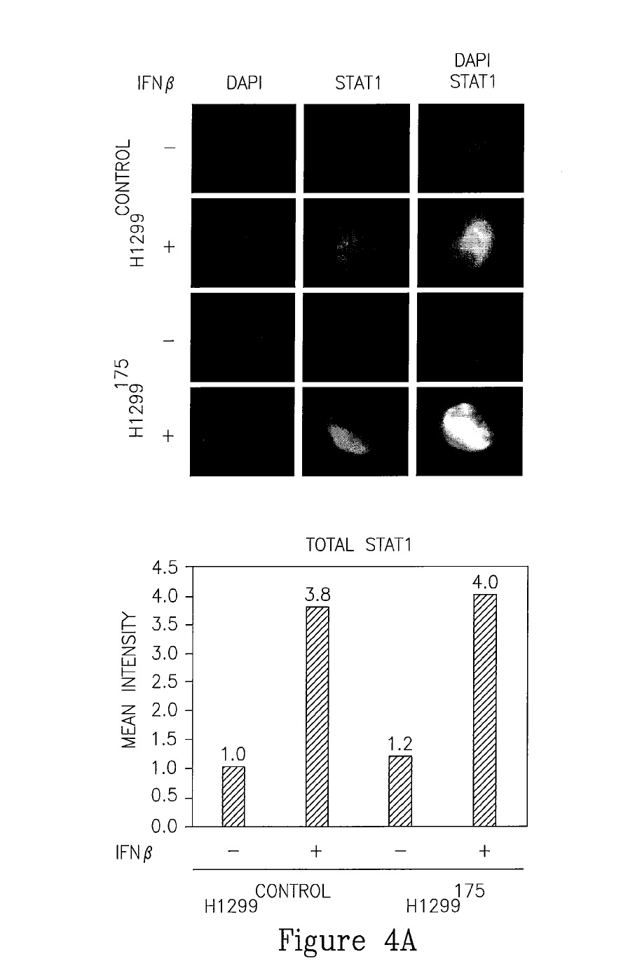

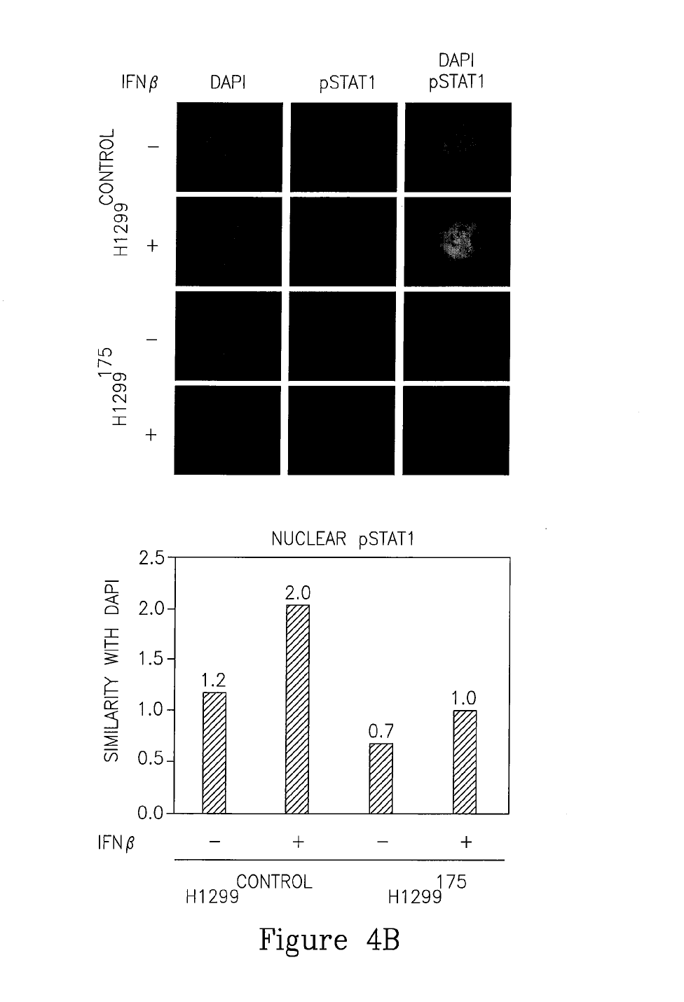

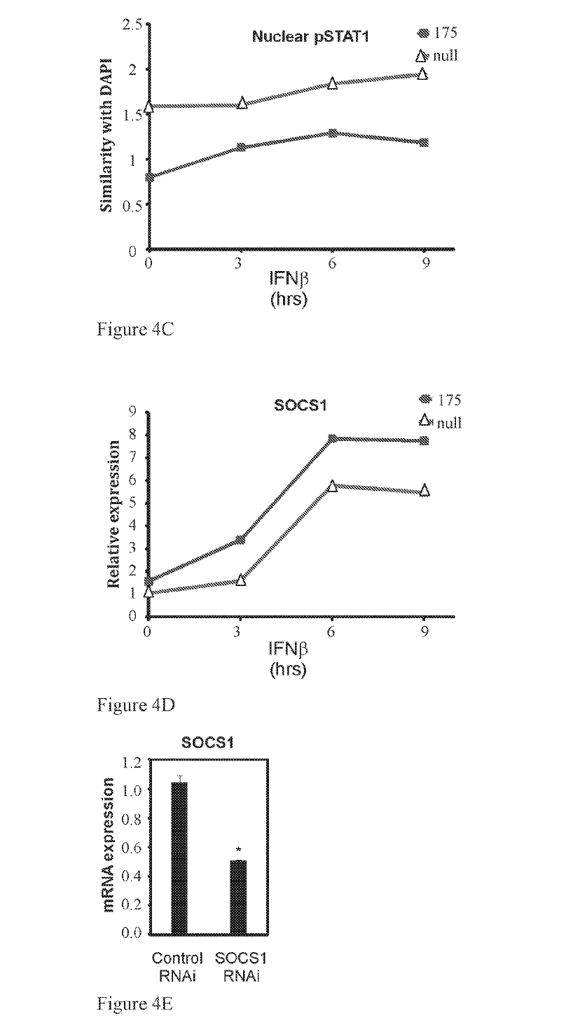

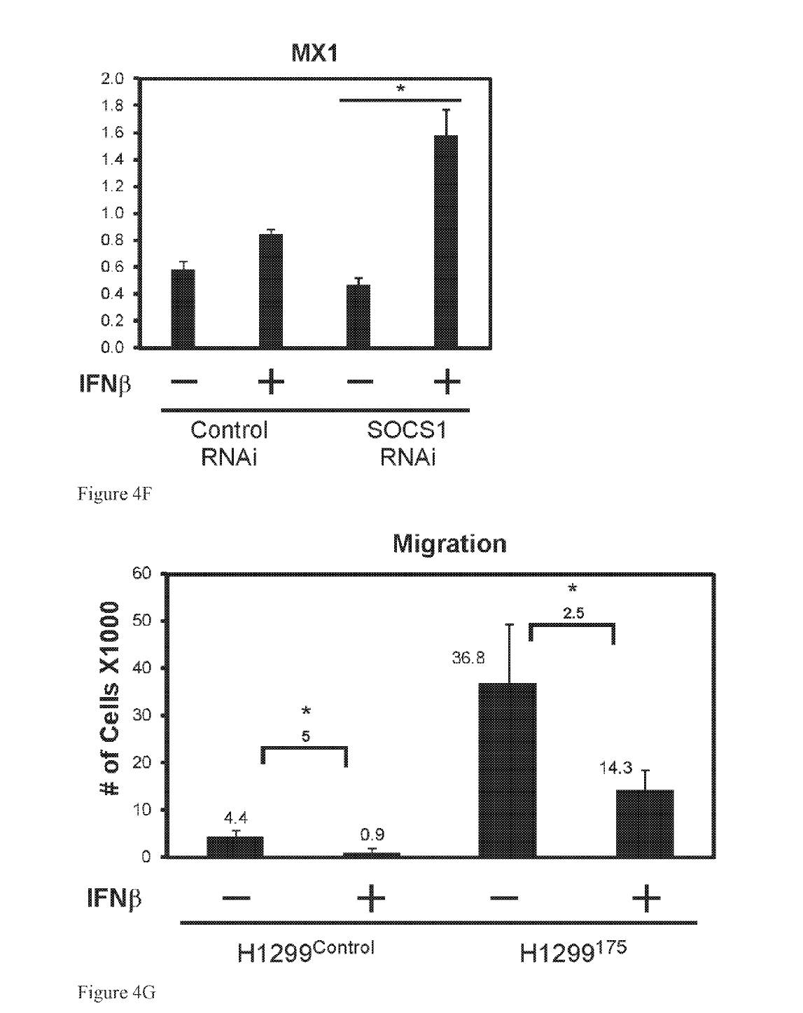

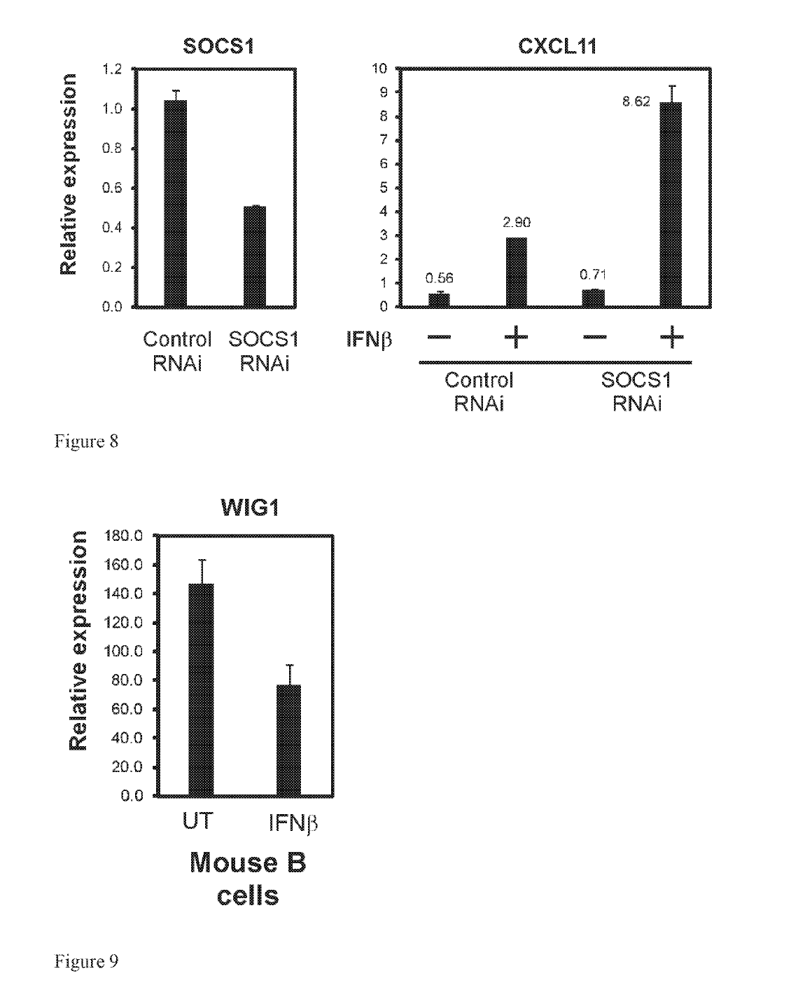

In order to investigate the effect of mutant p53 in cancer cells on the surrounding fibroblasts, the micro-array data obtained from the sorted H1299 were analyzed. Over-viewing differentially expressed IFN targets in H1299 that were grown alone or cultivated with CAFs, 3 major expression patterns were revealed: (i) responsiveness, namely both p53 null and mutant p53 bearing cells induced known IFN targets in a comparable manner, (ii) over-induction, in which IFN targets were highly induced by mutant p53 cells and (iii) attenuation, where IFN targets-induction was mitigated by mutant p53. Genes were identified by using one or more genes from each pattern as a bait vector and searched for other genes that exhibited a Pearson correlation of at least 0.9 to the bait vector, using a custom Matlab script (FIG. 3A). Next, the frequency of IFN targets was evaluated in each pattern, using the Intefreome database (See IFN enrichment—FIG. 3A lower panels). Expression of a representative gene from each pattern was validated by QRT-PCR (FIG. 3B). Pattern 2 (‘over-induction’) consists of two known inhibitors of the IFN signaling pathway, namely MAP3K8 and SOCS1. Pattern 3 (‘attenuation’) on the other hand, consists of two known tumor inhibitors—NMI and MX1. To examine whether the effect of mutant p53 on IFN pathway is a general phenomenon, this effect was analyzed in lung cancer cell line A549 and SKBR3 breast cancer cells. MX1 exhibited the same expression patterns in these cell lines, indicating that mutant p53 averts IFNs pathways at large. Introducing the H1299 panel with recombinant IFNs α, β and γ, yielded similar expression patterns of MX1 (FIG. 3C-E). The observation that mutant p53 had a similar effect on MX1 expression upon administration of all the IFNs suggests that mutant p53 exerts its effect on IFNs downstream targets rather than interfering with IFN itself or with its up-stream effectors. All IFNs pathways converge into the JAK1-mediated phosphorylation of STAT1, suggesting that the JAK/STAT components are affected by mutant p53. To test whether mutant p53 hinders the expression and phosphorylation of STAT1, H1299 were treated with IFN-β and stained with antibodies against p53, STAT1 and phospho-STAT1 (pSTAT1). Cells were then fixed and analyzed with the Image stream FACS sorter which photographs each individual sorted cell, thereby allowing a thorough investigation of a plethora of parameters, such as sub-cellular localization of proteins for the entire cell population. Total STAT1 levels rose following IFN-β administration, however without an apparent difference between the control and H1299¹⁷⁵ (FIG. 4A). pSTAT1 was exclusively present in the nuclei of the p53 null cells following 16 h of IFN-β treatment (FIG. 4B). The same experimental setup was used with shorter time laps and revealed a continuous lower pSTAT1 levels in H1299¹⁷⁵ (FIG. 4C). JAK1 which phosphporylates STAT1 is known to be inhibited by SOCS1, as part of the IFN negative feedback loop. As SOCS1 belongs to the ‘over-induction’induction’ pattern exhibited by the mutant p53 cells, we measured its expression levels as well. SOCS1 exhibited a mirror image of pSTAT1, namely was elevated in H1299¹⁷⁵ during IFN-β treatment (FIG. 4D). To test whether SOCS1 mediates the inhibiting effect of mutant p53, SOCS1 expression was knocked down in H1299¹⁷⁵ (FIG. 4E). The cells were then exposed to IFN-β treatment and indeed the expression of IFN-β targets MX1 and CXCL11 was regained (FIG. 4F and FIG. 8. respectively). Mutant p53 is known to facilitate invasion and migration either by promoting EMT or by negating p63 inhibition on invasion-promoting pathways. In addition, IFNs have been reported to repress invasion of cancer cells via MX1. Therefore it was measured the effect of IFN-β on the migratory capacity of cancer cells. First, H1299¹⁷⁵ indeed proved to migrate more effectively than their p53 depleted counterparts (FIG. 4G). In addition, the ability of the latter to migrate was nullified in the presence of IFN-β. Notably, H1299¹⁷⁵ migratory ability was reduced upon IFN-β treatment, however to a lesser extent. In sum, mutant p53 is able to moderate IFN-β response by over activating SOCS1 and reducing the levels of pSTAT1, thus reducing the inhibiting effect of IFN-β on cell migration.

### Example 4

### IFN-β Attenuates Mutant p53 Levels Through Inhibition of its mRNA Stabilizer, WIG1

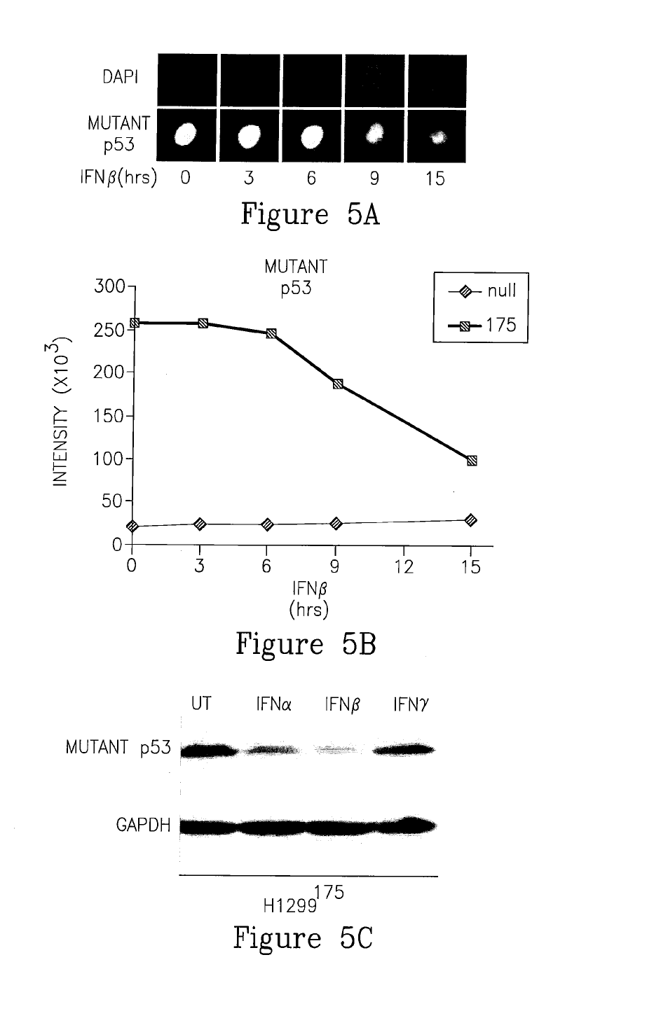

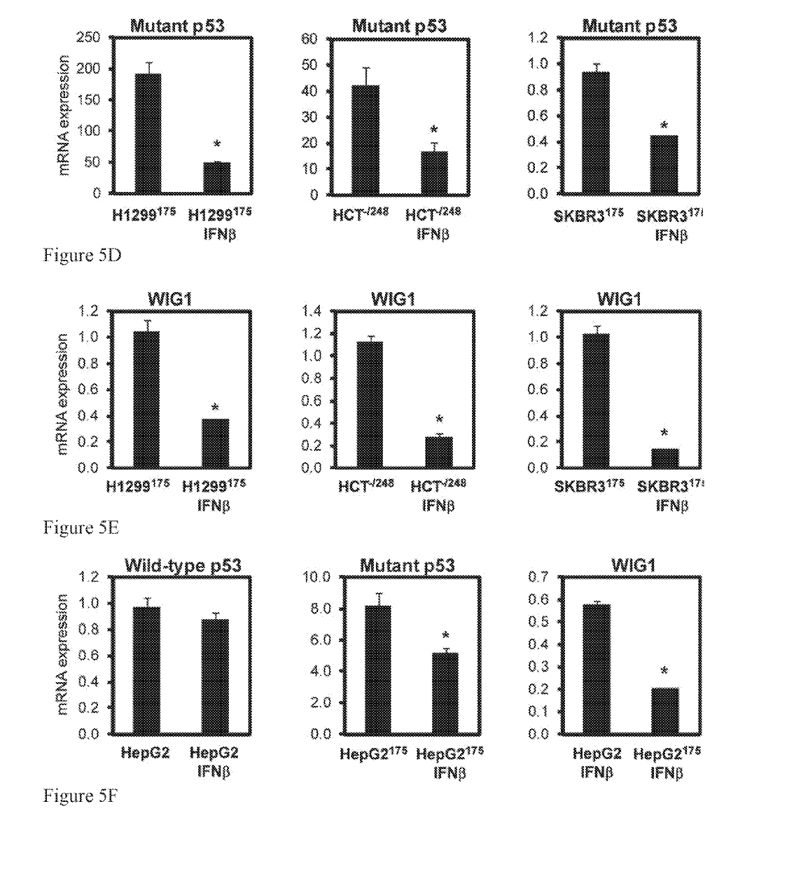

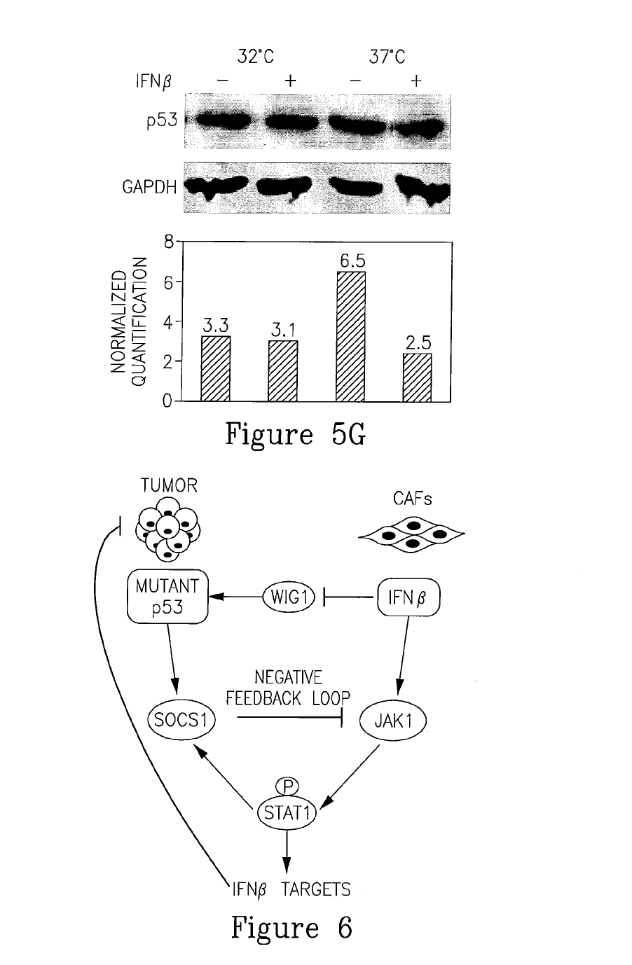

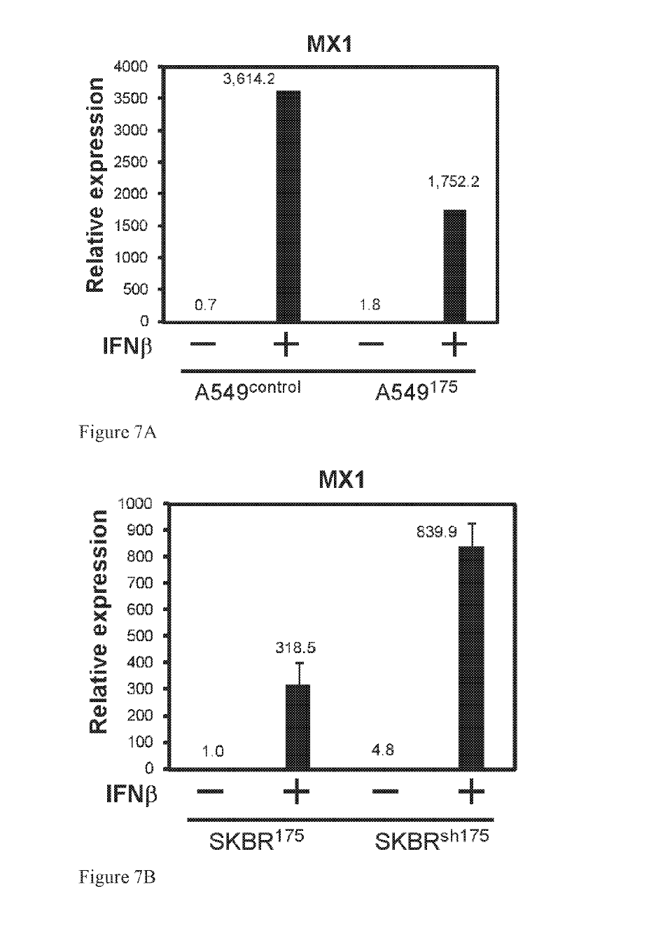

During the former set of experiments it was found that mutant p53 protein levels dramatically declined after 9 hours of IFN-β exposure (FIGS. 5A and B, Image stream analysis).

To verify this finding, all three IFNs were administered for 24 hours and western blotting was performed. Indeed, mutant p53 protein levels declined following IFN-α, β and γ treatment (FIG. 5C). QRT-PCR analysis revealed that mutant p53 RNA levels were reduced as well (FIG. 5D, left hand side). Since mutant p53 is expressed under the control of a viral promoter in the system described herein, the inventors wished to exclude the possibility that this observation stems from the anti-viral related effect of IFNs. For that purpose, two cell lines were utilized, which harbor endogenous p53 mutants, namely the HCT^{-/}248 knock-in cell line and SKBR3 cells which express endogenous p53^{R175H}. Notably, both cell lines exhibited a significant reduction in mutant p53 RNA levels upon IFN-β treatment (FIG. 5D, right hand side). These observations suggested that IFN-β compromises mutant p53 RNA stability.

Wild type p53 is instrumental for cell fate decisions and is therefore subjected to several tiers of control. One mode of regulation is exerted on its mRNA molecule in terms of stability and translation. WIG1 is a zinc finger protein capable of binding a U-rich element in the 3′ region of p53 mRNA, thereby inhibiting its de-adenylation and increasing its stability. As both wild type and mutant p53 mRNAs have identical 3′ sequences, mutant p53 benefits from WIG1 activity. Therefore it was examined whether WIG1 is affected by IFN-β. WIG1 levels decreased upon IFN-β treatment in all tested cell lines (FIG. 5E). These observations were not restricted to human cells as WIG1 down-regulation was also evident in mouse B-cells treated with IFN-β (FIG. 9, data retrieved from a micro-array by Chang et al. (2007).

Several studies documented a positive interaction between IFN-β and wild type p53 (Chiantore et al., 2012; Takaoka et al., 2003, among others), thus WIG1-mediated repression of wild type p53 by IFN-β seems to be counter-intuitive. While WIG1 is a bona fide target of wild type p53, mutant p53 seems to exert a dominant negative effect over its expression. Therefore a differential effect of IFN-β on mutant and wild type p53 RNA levels might be achieved by the wild type specific targeting of WIG1. As illustrated in FIG. 5F, HepG2 cells expressing either wild type or mutant p53 were subjected to IFN-β and only mutant p53 levels were reduced. Notably, WIG1 levels were significantly lower in the mutant p53 expressing cells. Thus, only wild type p53 can bypass the attenuating effect of IFN-β on WIG1 expression and maintain intact stable pool of mRNA.

In the experiment depicted in FIG. 5G, II1299 harboring a mutated Temperature Sensitive (TS) form of p53 were used. At 37° C., this form is at a mutated conformational state, whereas at 32° C. it shifts to a wild type conformation. This is a common system for comparing wild type and mutant p53, on an isogenic background. Indeed as shown in the figure by western blot, at 32° C., IFN-β had no effect on p53 levels, while at 37° C. it reduced mutant p53 protein levels by more than half.

### Example 5

### Sequences

Primer and probe sequences may be readily prepared by the skilled artisan based on p53 sequences known in the art. For example, the following sequences for human p53 have been disclosed:

Human p53 mRNA-accession no.: NM_{—}000546 (NM_{—}000546.5, transcript variant 1); SEQ ID NO: 2:

(SEQ ID NO: 2)
gatgggattg gggttttccc ctcccatgtg ctcaagactg gcgctaaaag ttttgagctt
ctcaaaagtc tagagccacc gtccagggag caggtagctg ctgggctccg gggacacttt
gcgttcgggc tgggagcgtg ctttccacga cggtgacacg cttccctgga ttggcagcca
gactgccttc cgggtcactg ccatggagga gccgcagtca gatcctagcg tcgagccccc
tctgagtcag gaaacatttt cagacctatg gaaactactt cctgaaaaca acgttctgtc
ccccttgccg tcccaagcaa tggatgattt gatgctgtcc ccggacgata ttgaacaatg
gttcactgaa gacccaggtc cagatgaagc tcccagaatg ccagaggctg ctccccccgt
ggcccctgca ccagcagctc ctacaccggc ggcccctgca ccagccccct cctggcccct
gtcatcttct gtcccttccc agaaaaccta ccagggcagc tacggtttcc gtctgggctt
cttgcattct gggacagcca agtctgtgac ttgcacgtac tcccctgccc tcaacaagat
gttttgccaa ctggccaaga cctgccctgt gcagctgtgg gttgattcca cacccccgcc
cggcacccgc gtccgcgcca tggccatcta caagcagtca cagcacatga cggaggttgt
gaggcgctgc ccccaccatg agcgctgctc agatagcgat ggtctggccc ctcctcagca
tcttatccga gtggaaggaa atttgcgtgt ggagtatttg gatgacagaa acacttttcg
acatagtgtg gtggtgccct atgagccgcc tgaggttggc tctgactgta ccaccatcca
ctacaactac atgtgtaaca gttcctgcat gggcggcatg aaccggaggc ccatcctcac
catcatcaca ctggaagact ccagtggtaa tctactggga cggaacagct ttgaggtgcg
tgtttgtgcc tgtcctggga gagaccggcg cacagaggaa gagaatctcc gcaagaaagg
ggagcctcac cacgagctgc ccccagggag cactaagcga gcactgccca acaacaccag
ctcctctccc cagccaaaga agaaaccact ggatggagaa tatttcaccc ttcagatccg
tgggcgtgag cgcttcgaga tgttccgaga gctgaatgag gccttggaac tcaaggatgc
ccaggctggg aaggagccag gggggagcag ggctcactcc agccacctga agtccaaaaa
gggtcagtct acctcccgcc ataaaaaact catgttcaag acagaagggc ctgactcaga
ctgacattct ccacttcttg ttccccactg acagcctccc acccccatct ctccctcccc
tgccattttg ggttttgggt ctttgaaccc ttgcttgcaa taggtgtgcg tcagaagcac
ccaggacttc catttgcttt gtcccggggc tccactgaac aagttggcct gcactggtgt
tttgttgtgg ggaggaggat ggggagtagg acataccagc ttagatttta aggtttttac
tgtgagggat gtttgggaga tgtaagaaat gttcttgcag ttaagggtta gtttacaatc
agccacattc taggtagggg cccacttcac cgtactaacc agggaagctg tccctcactg
ttgaattttc tctaacttca aggcccatat ctgtgaaatg ctggcatttg cacctacctc
acagagtgca ttgtgagggt taatgaaata atgtacatct ggccttgaaa ccacctttta
ttacatgggg tctagaactt gacccccttg agggtgcttg ttccctctcc ctgttggtcg
gtgggttggt agtttctaca gttgggcagc tggttaggta gagggagttg tcaagtctct
gctggcccag ccaaaccctg tctgacaacc tcttggtgaa ccttagtacc taaaaggaaa
tctcacccca tcccacaccc tggaggattt catctcttgt atatgatgat ctggatccac
caagacttgt tttatgctca gggtcaattt cttttttctt tttttttttt ttttttcttt
ttctttgaga ctgggtctcg ctttgttgcc caggctggag tggagtggcg tgatcttggc
ttactgcagc ctttgcctcc ccggctcgag cagtcctgcc tcagcctccg gagtagctgg
gaccacaggt tcatgccacc atggccagcc aacttttgca tgttttgtag agatggggtc
tcacagtgtt gcccaggctg gtctcaaact cctgggctca ggcgatccac ctgtctcagc
ctcccagagt gctgggatta caattgtgag ccaccacgtc cagctggaag ggtcaacatc
ttttacattc tgcaagcaca tctgcatttt caccccaccc ttcccctcct tctccctttt
tatatcccat ttttatatcg atctcttatt ttacaataaa actttgctgc cacctgtgtg
tctgaggggt g

The corresponding polypeptide sequence is (SEQ ID NO: 3):

(SEQ ID NO: 3)
MEEPQSDPSV EPPLSQETFS DLWKLLPENN VLSPLPSQAM
DDLMLSPDDI EQWFTEDPGP DEAPRMPEAA PPVAPAPAAP
TPAAPAPAPS WPLSSSVPSQ KTYQGSYGFR LGFLHSGTAK
SVTCTYSPAL NKMFCQLAKT CPVQLWVDST PPPGTRVRAM
AIYKQSQHMT EVVRRCPHHE RCSDSDGLAP PQHLIRVEGN
LRVEYLDDRN TFRHSVVVPY EPPEVGSDCT TIHYNYMCNS
SCMGGMNRRP ILTIITLEDS SGNLLGRNSF EVRVCACPGR
DRRTEEENLR KKGEPHHELP PGSTKRALPN NTSSSPQPKK
KPLDGEYFTL QIRGRERFEM FRELNEALEL KDAQAGKEPG
GSRAHSSHLK SKKGQSTSRH KKLMFKTEGP DS

It is to be understood that such primer and probe sequences may be altered based on known or identified genetic variations in the p53 gene, as known in the art. For example, splicing variants of the human P53 gene have been described, generated by alternative splicing, use of alternative translation site or alternative promoter. As these isoforms typically share a common DNA-binding domain (DBD), such isoform sequences may be detected by certain embodiments of the methods of the invention.

Exemplary sequences of human type I IFNs are:

Human IFN beta 1, fibroblast (IFNB1), mRNA-accession no. NM_{—}002176.2 (SEQ ID NO: 4):

(SEQ ID NO: 4)
acattctaac tgcaaccttt cgaagccttt gctctggcac
aacaggtagt aggcgacact gttcgtgttg tcaacatgac
caacaagtgt ctcctccaaa ttgctctcct gttgtgcttc
tccactacag ctctttccat gagctacaac ttgcttggat
tcctacaaag aagcagcaat tttcagtgtc agaagctcct
gtggcaattg aatgggaggc ttgaatactg cctcaaggac
aggatgaact ttgacatccc tgaggagatt aagcagctgc
agcagttcca gaaggaggac gccgcattga ccatctatga
gatgctccag aacatctttg ctattttcag acaagattca
tctagcactg gctggaatga gactattgtt gagaacctcc
tggctaatgt ctatcatcag ataaaccatc tgaagacagt
cctggaagaa aaactggaga aagaagattt caccagggga
aaactcatga gcagtctgca cctgaaaaga tattatggga
ggattctgca ttacctgaag gccaaggagt acagtcactg
tgcctggacc atagtcagag tggaaatcct aaggaacttt
tacttcatta acagacttac aggttacctc cgaaactgaa
gatctcctag cctgtgcctc tgggactgga caattgcttc
aagcattctt caaccagcag atgctgttta agtgactgat
ggctaatgta ctgcatatga aaggacacta gaagattttg
aaatttttat taaattatga gttattttta tttatttaaa
ttttattttg gaaaataaat tatttttggt gcaaaagtca.

The corresponding polypeptide sequence (human IFN-β1 precursor) is set forth in SEQ ID NO: 5:

(SEQ ID NO: 5)
MTNKCLLQIA LLLCFSTTAL SMSYNLLGFL QRSSNFQCQK
LLWQLNGRLE YCLKDRMNFD IPEEIKQLQQ FQKEDAALTI
YEMLQNIFAI FRQDSSSTGW NETIVENLLA NVYHQINHLK
TVLEEKLEKE DFTRGKLMSS LHLKRYYGRI LHYLKAKEYS
HCAWTIVRVE ILRNFYFINR LTGYLRN.

The mature IFN-β polypeptide (after cleavage of the 21 amino acid signal peptide) corresponds to positions 22-187 of SEQ ID NO: 5, and is set forth in SEQ ID NO: 1.

Human IFN alpha 1 (IFNA1), mRNA-accession no. NM_{—}024013.2 (SEQ ID NO: 6):

(SEQ ID NO: 6)
caaggttcag agtcacccat ctcagcaagc ccagaagtat
ctgcaatatc tacgatggcc tcgccctttg ctttactgat
ggtcctggtg gtgctcagct gcaagtcaag ctgctctctg
ggctgtgatc tccctgagac ccacagcctg gataacagga
ggaccttgat gctcctggca caaatgagca gaatctctcc
ttcctcctgt ctgatggaca gacatgactt tggatttccc
caggaggagt ttgatggcaa ccagttccag aaggctccag
ccatctctgt cctccatgag ctgatccagc agatcttcaa
cctctttacc acaaaagatt catctgctgc ttgggatgag
gacctcctag acaaattctg caccgaactc taccagcagc
tgaatgactt ggaagcctgt gtgatgcagg aggagagggt
gggagaaact cccctgatga atgcggactc catcttggct
gtgaagaaat acttccgaag aatcactctc tatctgacag
agaagaaata cagcccttgt gcctgggagg ttgtcagagc
agaaatcatg agatccctct ctttatcaac aaacttgcaa
gaaagattaa ggaggaagga ataacatctg gtccaacatg
aaaacaattc ttattgactc atacaccagg tcacgctttc
atgaattctg tcatttcaaa gactctcacc cctgctataa
ctatgaccat gctgataaac tgatttatct atttaaatat
ttatttaact attcataaga tttaaattat ttttgttcat
ataacgtcat gtgcaccttt acactgtggt tagtgtaata
aaacatgttc cttatattta ctc.

The corresponding polypeptide sequence, (human IFN-α1 precursor) is set forth in SEQ ID NO: 7 below. The mature IFN-α1 polypeptide corresponds to positions 24-189 of SEQ ID NO: 7.

(SEQ ID NO: 7)
MASPFALLMV LVVLSCKSSC SLGCDLPETH SLDNRRTLML
LAQMSRISPS SCLMDRHDFG FPQEEFDGNQ FQKAPAISVL
HELIQQIFNL FTTKDSSAAW DEDLLDKFCT ELYQQLNDLE
ACVMQEERVG ETPLMNADSI LAVKKYFRRI TLYLTEKKYS
PCAWEVVRAE IMRSLSLSTN LQERLRRKE.

Human IFN alpha 2 (IFNA2), mRNA-accession no. NM_{—}000605.3 (SEQ ID NO: 8):

(S43Q ID NO: 8)
gagaacctgg agcctaaggt ttaggctcac ccatttcaac
cagtctagca gcatctgcaa catctacaat ggccttgacc
tttgctttac tggtggccct cctggtgctc agctgcaagt
caagctgctc tgtgggctgt gatctgcctc aaacccacag
cctgggtagc aggaggacct tgatgctcct ggcacagatg
aggagaatct ctcttttctc ctgcttgaag gacagacatg
actttggatt tccccaggag gagtttggca accagttcca
aaaggctgaa accatccctg tcctccatga gatgatccag
cagatcttca atctcttcag cacaaaggac tcatctgctg
cttgggatga gaccctccta gacaaattct acactgaact
ctaccagcag ctgaatgacc tggaagcctg tgtgatacag
ggggtggggg tgacagagac tcccctgatg aaggaggact
ccattctggc tgtgaggaaa tacttccaaa gaatcactct
ctatctgaaa gagaagaaat acagcccttg tgcctgggag
gttgtcagag cagaaatcat gagatctttt tctttgtcaa
caaacttgca agaaagttta agaagtaagg aatgaaaact
ggttcaacat ggaaatgatt ttcattgatt cgtatgccag
ctcacctttt tatgatctgc catttcaaag actcatgttt
ctgctatgac catgacacga tttaaatctt ttcaaatgtt
tttaggagta ttaatcaaca ttgtattcag ctcttaaggc
actagtccct tacagaggac catgctgact gatccattat
ctatttaaat atttttaaaa tattatttat ttaactattt
ataaaacaac ttatttttgt tcatattatg tcatgtgcac
ctttgcacag tggttaatgt aataaaatat gttctttgta
tttggtaaat ttattttgtg ttgttcattg aacttttgct
atggaaactt ttgtacttgt ttattcttta aaatgaaatt
ccaagcctaa ttgtgcaacc tgattacaga ataactggta
cacttcattt atccatcaat attatattca agatataagt
aaaaataaac tttctgtaaa cca.

The corresponding polypeptide sequence (human IFN-α2 precursor) is set forth in SEQ ID NO: 9 below. The mature IFN-α2 polypeptide corresponds to positions 24-188 of SEQ ID NO: 9, and is set forth in SEQ ID NO: 40.

(SEQ ID NO: 9)
MALTFALLVA LLVLSCKSSC SVGCDLPQTH SLGSRRTLML
LAQMRRISLF SCLKDRHDFG FPQEEFGNQF QKAETIPVLH
EMIQQIFNLF STKDSSAAWD ETLLDKFYTE LYQQLNDLEA
CVIQGVGVTE TPLMKEDSIL AVRKYFQRIT LYLKEKKYSP
CAWEVVRAEI MRSFSLSTNL QESLRSKE.

The sequence of human IFN gamma (type II IFN) may be found at accession no. NM_{—}000619.2.

Additional IFN sequences are known in the art and are available at Genebank. Additional IFN polypeptides employed in embodiments of the invention have at least 90% amino acid homology to the above sequences, such as polypeptides comprising conservative amino acid substitutions or other silent mutations (e.g. 1-5 or up to 10 substitutions). For example, BETASERON marketed by BAYER is a recombinant human IFN-β (IFN-β-1b), in which cysteine 17 is substituted with serine (C17S).

### Example 6

### YNS, an IFN-α Analog, Reduces Mutant p53 Levels

In the experiments described herein, H1299 cells (either p53 null or expressing mutant p53 R175H), were treated with IFN-β, or with YNS. The latter is an IFN-α2 analog, which differs from native human IFN-α2 (SEQ ID NO: 40) by substitution of the histidine at position 57 (H57) to tyrosine, substitution of the glutamate at position 58 (E58) to asparagine, and substitution of the glutamine at position 61 (Q61) to serine.

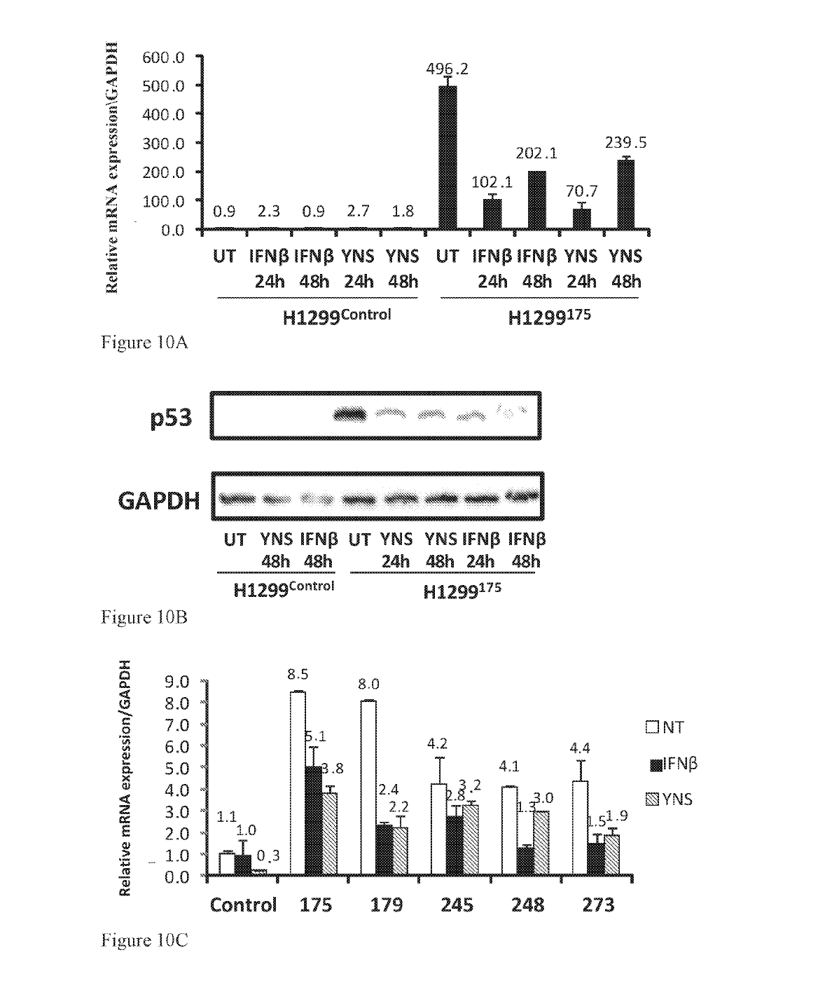

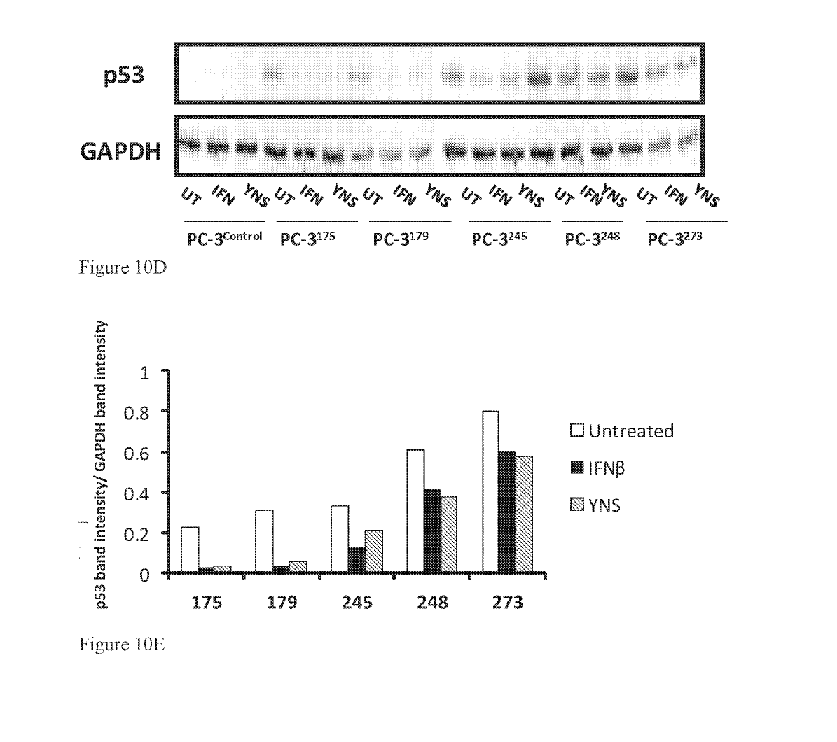

As can be seen in FIGS. 10A and B, both IFN-β and YNS induced a reduction in mutant p53 levels, seen both in the mRNA and protein levels.

Next, a panel of prostate carcinoma PC-3 sub-lines expressing different mutant p53 forms were treated with IFN-β or YNS. As can be seen in FIG. 10C-E, a reduction in p53 levels was observed, wherein the most prominent reduction was seen in conformational mutations R175H and H179R.

### REFERENCES

1. Arai et al, “Immunologically distinct p53 molecules generated by alternative splicing” Mol Cell Biol 6:3232-3239 (1986).
2. Bykov V J, Issaeva N, Zache N, Shilov A, Hultcrantz M, et al. (2005) Reactivation of mutant p53 and induction of apoptosis in human tumor cells by maleimide analogs. J Biol Chem 280: 30384-30391.
3. Chang W L, Coro E S, Rau F C, Xiao Y, Erle D J, et al. (2007) Influenza virus infection causes global respiratory tract B cell response modulation via innate immune signals J Immunol 178: 1457-1467.
4. Chiantore M V, Vannucchi S, Accardi R, Tommasino M, Percario Z A, et al. (2012) Interferon-beta induces cellular senescence in cutaneous human papilloma virus-transformed human keratinocytes by affecting p53 transactivating activity. PLoS One 7: e36909.
5. Dunn G P, Koebel C M, Schreiber R D (2006) Interferons, immunity and cancer immunoediting. Nat Rev Immunol 6: 836-848.
6. Foster B A, Coffey H A, Morin M J, Rastinejad F (1999) Pharmacological rescue of mutant p53 conformation and function. Science 286: 2507-2510.
7. Juang et al. J Interferon Cytokine Res. 2004 April; 24(4):231-43.
8. Levy et al., Current Opinion in Virology, 2011, 1:476-486.
9. Noguchi et al. International Journal of Oncology 32: 193-199, 2008.
10. Porta et al., Oncogene. 2005 Jan. 20; 24(4):605-15.
11. Samuels-Lev Y, O'Connor D J, Bergamaschi D, Trigiante G, Hsieh J K, et al. (2001) ASPP proteins specifically stimulate the apoptotic function of p53. Molecular Cell 8: 781-794.
12. Solomon H, Madar S, Rotter V (2011) Mutant p53 gain of function is interwoven into the hallmarks of cancer. J Pathol 225: 475-478.
13. Studeny M, Marini F C, Dembinski J L, Zompetta C, Cabreira-Hansen M, et al. (2004) Mesenchymal stem cells: potential precursors for tumor stroma and targeted-delivery vehicles for anticancer agents. J Natl Cancer Inst 96: 1593-1603.
14. Takaoka A, Hayakawa S, Yanai H, Stoiber D, Negishi H, et al. (2003) Integration of interferon-alpha/beta signalling to p53 responses in tumour suppression and antiviral defence. Nature 424: 516-523.
15. Tjandra S S, Hsu C, Goh Y I, Gurung A, Poon R, et al. (2007) IFN-{beta} signaling positively regulates tumorigenesis in aggressive fibromatosis, potentially by modulating mesenchymal progenitors. Cancer Res 67: 7124-7131.
16. Tsai Y C, Pestka S, Wang L H, Runnels L W, Wan S, et al. (2011) Interferon-beta signaling contributes to Ras transformation. PLoS One 6: e24291.
17. Wang, P. L., Sait, F. & Winter, G. The ‘wildtype’ conformation of p53: epitope mapping using hybrid proteins. Oncogene 20, 2318-2324 (2001).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

What is claimed is:

1. A method of treating or preventing a neoplastic disorder in a subject identified as expressing mutant p53 that is characterized by one or more mutations residing within the DNA binding domain, the method comprising administering to said subject type I IFN, thereby treating or preventing a neoplastic disorder in said subject.

2. The method according to claim 1, wherein determining whether the subject expresses mutant p53 is performed by determining the presence of mutant p53 protein or of mutant p53 mRNA transcript.

3. The method according to claim 1, wherein determining whether the subject expresses mutant p53 is performed by determining the presence of mutant p53 mRNA, wherein if said subject expresses mutant p53 mRNA, said subject is determined to be amenable for treatment with the type I IFN.

4. The method according to claim 1, wherein determining whether the subject expresses mutant p53 further comprises determining mutant p53 mRNA levels, wherein if said subject expresses mutant p53 mRNA, and the level of the mutant p53 mRNA is reduced upon type I IFN treatment, said subject is determined to be amenable for treatment with said type I IFN.

5. The method according to claim 1, wherein the subject is afflicted with or is predisposed to developing the neoplastic disorder.

6. The method according to claim 5, wherein said disorder is Li-Fraumeni syndrome or another disorder associated with a p53 germline mutation.

7. The method according to claim 5, wherein said disorder is cancer or cancer metastasis.

8. The method according to claim 5, wherein said subject is a cancer patient in a state of remission.

9. (canceled)

10. The method according to claim 1 wherein said type I IFN is formulated for in vivo administration, or wherein said type I IFN is formulated for ex vivo administration.

11. The method according to claim 1, wherein said type I IFN reduces cellular levels of mutant p53, thereby treating said subject.

12. The method according to claim 11 for reducing cellular levels of mutant p53, thereby sensitizing a chemotherapy-resistant tumor to chemotherapy, treating or preventing cancer or metastasis, or treating Li-Fraumeni syndrome in said subject.

13. The method according to claim 1, wherein said type I IFN is formulated as a pharmaceutical composition in unit dosage form, at a dose that does not substantially induce apoptotic cell death when administered to said subject.

14. The method according to claim 1, wherein said IFN is an IFN-β or an IFN-α.

15. The method according to claim 14, wherein said IFN is IFN-β, preferably IFN-β1.

16. The method according to claim 14, wherein said IFN is IFN-α, preferably IFN-α2.

17. The method according to claim 1, wherein said type I IFN is the sole active ingredient.

18. A nucleic acid construct for use in the treatment or prevention of a neoplastic disorder,
wherein:
the construct comprises a nucleic acid sequence encoding a type I IFN, which nucleic acid sequence is operably linked to a transcription-regulating sequence, and
the subject is identified as expressing mutant p53 that is characterized by one or more mutations residing within the DNA binding domain.

19. A method for determining whether a subject is amenable for treatment with a type I interferon (IFN), comprising the steps of:
a) determining whether the subject expresses mutant p53 characterized by one or more mutations residing within the DNA binding domain; and
b) if the subject expresses the mutant p53, identifying said subject as amenable for treatment with the type I IFN;
wherein said treatment with said type I IFN is used for reducing cellular levels of mutant p53.

20. The method of claim 19, wherein the type I IFN is an IFN-β or an IFN-α.

21. The method of claim 19, wherein determining whether the subject expresses mutant p53 is performed by determining the presence of mutant p53 mRNA transcript or of mutant p53 protein levels.

22-54. (canceled)


SEQUENCE LISTING (42 sequences)

SEQ ID NO: 1 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 2 - DNA, 2591 bp, Homo sapiens
gatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 3 - protein, 392 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDS

SEQ ID NO: 4 - DNA, 840 bp, Homo sapiens
acattctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtca

SEQ ID NO: 5 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 6 - DNA, 863 bp, Homo sapiens
caaggttcagagtcacccatctcagcaagcccagaagtatctgcaatatctacgatggcctcgccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataacatctggtccaacatgaaaacaattcttattgactcatacaccaggtcacgctttcatgaattctgtcatttcaaagactctcacccctgctataactatgaccatgctgataaactgatttatctatttaaatatttatttaactattcataagatttaaattatttttgttcatataacgtcatgtgcacctttacactgtggttagtgtaataaaacatgttccttatatttactc

SEQ ID NO: 7 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 8 - DNA, 1143 bp, Homo sapiens
gagaacctggagcctaaggtttaggctcacccatttcaaccagtctagcagcatctgcaacatctacaatggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaaaactggttcaacatggaaatgattttcattgattcgtatgccagctcacctttttatgatctgccatttcaaagactcatgtttctgctatgaccatgacacgatttaaatcttttcaaatgtttttaggagtattaatcaacattgtattcagctcttaaggcactagtcccttacagaggaccatgctgactgatccattatctatttaaatatttttaaaatattatttatttaactatttataaaacaacttatttttgttcatattatgtcatgtgcacctttgcacagtggttaatgtaataaaatatgttctttgtatttggtaaatttattttgtgttgttcattgaacttttgctatggaaacttttgtacttgtttattctttaaaatgaaattccaagcctaattgtgcaacctgattacagaataactggtacacttcatttatccatcaatattatattcaagatataagtaaaaataaactttctgtaaacca

SEQ ID NO: 9 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 10 - DNA, 24 bp, Artificial Sequence
  IFN alpha forward primer
ccctctctttatcaacaaacttgc

SEQ ID NO: 11 - DNA, 21 bp, Artificial Sequence
  IFN alpha reverse primer
ttgttttcatgttggaccaga

SEQ ID NO: 12 - DNA, 20 bp, Artificial Sequence
  IFN beta forward primer
cgacactgttcgtgttgtca

SEQ ID NO: 13 - DNA, 20 bp, Artificial Sequence
  IFN beta reverse primer
gaagcacaacaggagagcaa

SEQ ID NO: 14 - DNA, 21 bp, Artificial Sequence
  MX1 forward primer
gtttccgaagtggacatcgca

SEQ ID NO: 15 - DNA, 23 bp, Artificial Sequence
  MX1 reverse primer
ccattcagtaatagagggtggga

SEQ ID NO: 16 - DNA, 23 bp, Artificial Sequence
  STAT1 forward primer
atgtctcagtggtacgaacttca

SEQ ID NO: 17 - DNA, 23 bp, Artificial Sequence
  STAT1 reverse primer
tgctccaggaattttgagtcaag

SEQ ID NO: 18 - DNA, 19 bp, Artificial Sequence
  SOCS1 forward primer
cccctggttgttgtagcag

SEQ ID NO: 19 - DNA, 20 bp, Artificial Sequence
  SOCS1 reverse primer
gtaggaggtgcgagttcagg

SEQ ID NO: 20 - DNA, 21 bp, Artificial Sequence
  p53 forward primer
cccaagcaatggatgatttga

SEQ ID NO: 21 - DNA, 21 bp, Artificial Sequence
  p53 reverse primer
ggcattctgggagcttcatct

SEQ ID NO: 22 - DNA, 21 bp, Artificial Sequence
  WIG1 forward primer
cggcagagaattccacgtgat

SEQ ID NO: 23 - DNA, 21 bp, Artificial Sequence
  WIG1 reverse primer
atctcttcgccagctccaaca

SEQ ID NO: 24 - DNA, 21 bp, Artificial Sequence
  GFP forward primer
gggcacaagctggagtacaac

SEQ ID NO: 25 - DNA, 21 bp, Artificial Sequence
  GFP reverse primer
caccttgatgccgttcttctg

SEQ ID NO: 26 - DNA, 20 bp, Artificial Sequence
  dsRed forward primer
gctcctccaagaacgtcatc

SEQ ID NO: 27 - DNA, 20 bp, Artificial Sequence
  dsRed forward primer
gggtgcttcacgtacacctt

SEQ ID NO: 28 - DNA, 25 bp, Artificial Sequence
  SP1 forward primer
aaacgtacacacacaggtgagaaga

SEQ ID NO: 29 - DNA, 21 bp, Artificial Sequence
  SP1 reverse primer
ctcctcatgaagcgcttagga

SEQ ID NO: 30 - DNA, 21 bp, Artificial Sequence
  p21 forward primer
ggcagaccagcatgacagatt

SEQ ID NO: 31 - DNA, 19 bp, Artificial Sequence
  p21 reverse primer
gcggattagggcttcctct

SEQ ID NO: 32 - DNA, 19 bp, Artificial Sequence
  ELN forward primer
gcaggagttaagcccaagg

SEQ ID NO: 33 - DNA, 20 bp, Artificial Sequence
  ELN reverse primer
tgtagggcagtccatagcca

SEQ ID NO: 34 - DNA, 20 bp, Artificial Sequence
  PDGFB forward primer
ttaagaaggccacggtgacg

SEQ ID NO: 35 - DNA, 20 bp, Artificial Sequence
  PDGFB reverse primer
tctcacacttgcatgccagg

SEQ ID NO: 36 - DNA, 20 bp, Artificial Sequence
  CXCL11 forward primer
gacgctgtctttgcataggc

SEQ ID NO: 37 - DNA, 23 bp, Artificial Sequence
  CXCL11 reverse primer
ggatttaggcatcgttgtccttt

SEQ ID NO: 38 - DNA, 21 bp, Artificial Sequence
  IFIT1 forward primer
aagctttcaaatcccttccgc

SEQ ID NO: 39 - DNA, 20 bp, Artificial Sequence
  IFIT1 reverse primer
gccttggcccgttcataatt

SEQ ID NO: 40 - protein, 165 aa, Homo sapiens [sequence omitted]

SEQ ID NO: 41 - protein, 7 aa, Artificial Sequence
  synthetic peptide
KRLKSKE

SEQ ID NO: 42 - protein, 6 aa, Artificial Sequence
  synthetic peptide
KRLKSK